(19) United States
(12) Patent Application Publication
(10) Pub. No.: US 2023/0136569 A1
(43) Pub. Date: May 4, 2023

(54) USE OF NMN TO REDUCE IMMUNODEPRESSION AND IMMUNOSENESCENCE

(71) Applicant: NUVAMID SA, Lausanne (CH)

(72) Inventors: Guillaume BERMOND, MARSEILLE (FR); Laurent GARCON, SAUSSET LES PINS (FR)

(73) Assignee: NUVAMID SA, Lausanne (CH)

(21) Appl. No.: 17/927,478

(22) PCT Filed: May 26, 2021
(86) PCT No.: PCT/EP2021/064121

§ 371 Date: Nov. 23, 2022

(30) Foreign Application Priority Data
May 29, 2020 (FR) FR2005739

(51) Int. Cl. A61K 31/706; A61K 31/6615

(52) U.S. Cl. CPC A61K 31/706; A61K 31/6615

(57) ABSTRACT

The invention pertains to nicotinamide mononucleotide, a pharmaceutically acceptable derivative thereof, a pharmaceutically acceptable precursor thereof, or a pharmaceutically acceptable salt thereof, for use thereof in decreasing immunosenescence and/or for improving immune response to vaccination, and to compositions comprising the same.

### FIELD OF THE INVENTION

The present invention pertains to the use of NMN, a pharmaceutically acceptable precursor thereof, a pharmaceutically acceptable derivative thereof, or a pharmaceutically acceptable salt thereof, for the treatment and prevention of immunodeficiency and particularly of immunosenescence. The present invention also pertains to compositions comprising NMN, a pharmaceutically acceptable precursor thereof, a pharmaceutically acceptable derivative thereof, or a pharmaceutically acceptable salt thereof, for the treatment and prevention of immunodeficiency and more particularly immunosenescence.

### TECHNICAL BACKGROUND

Immunodeficiency is the phenomenon of reduced primary immune response of an individual. For example, an immunocompromised individual may not have satisfactory defensive response against bacterial and viral infections. Immunodeficiency can either be of hereditary origin, or caused by immunosuppressive treatment, or caused by a disease such as cancer, HIV infection or ageing of the individual.

Immunosenescence is a form of immunodeficiency and is the phenomenon of marked loss of the efficacy of the immune system, whether innate and/or adaptive immunity, induced by ageing of the individual. Immunosenescence contributes towards increased sensitivity of elderly persons to viral and bacterial infections in particular. The causes of immunosenescence are multiple. Immunosenescence can be caused by ageing, disease such as cancer or osteoporosis, smoking, diet, pollution and other environmental factors.

Immunosenescence affects various types of cells in bone marrow and the thymus, mature lymphocytes in peripheral blood and secondary lymphatic organs, as well as elements of the innate immune system. The immune system can be divided into an innate part chiefly composed of monocytes, natural killer cells (NK) and dendritic cells (DC), and an adaptive part represented by B and T lymphocytes. Innate immunity is better preserved, while more serious changes that are often harmful and age-dependent, occur in the adaptive immune system. Among the other changes brought by immunosenescence, mention can be made of involution of the thymus characterized by a reduction in the overall size of the organ and replacement of the functional cortex and medullar tissue by fat, a reduction in the number of naive T and B cells leaving the thymus and the variety of immune cells. Naive cells are T or B cells that have not yet encountered an antigen. Immunosenescence leads to less effective lymphocyte production, activation and reactivity. The result is increased sensitivity to inflammatory reactions and autoimmunity, and diminished reactions to infections, tumours and vaccinations.

In addition, the immunological changes induced by ageing translate as the onset of low-level chronic inflammation known as «inflammaging». This inflammation is associated with release in large amounts of pro-inflammatory agents and of proteases into the tissues and in particular into the mucosa, leading to inflammaging. Inflammaging can be defined as inflammation not exhibiting any conventional clinical sign of inflammation, namely redness, swelling, heat, pain. However, a progressive increase is ascertained in markers of inflammation such as interleukin-6 (IL-6), tumour necrosis factor alpha (TNF-α), interleukin 1 antagonist receptor (IL-1Ra), C-reactive protein (CRP). This inflammaging is found in elderly persons and mobilises the immune system which is no longer able to defend the body in adapted manner against the intrusion of bacteria, fungi or viruses. This raises obvious problems of public health.

All the immune and inflammatory changes associated with immunosenescence therefore raise obvious problems of public health. There is therefore a need to develop novel compositions allowing immunosenescence to be decreased.

### SUMMARY OF THE INVENTION

These objectives are reached by means of nicotinamide mononucleotide (NMN) and compositions comprising the same for use thereof in the prevention and/or treatment of immunodeficiency, preferably immunosenescence.

The subject of the present invention is nicotinamide mononucleotide (NMN), a pharmaceutically acceptable precursor thereof, a pharmaceutically acceptable derivative thereof, or a pharmaceutically acceptable salt thereof, for the prevention and/or treatment of immunosenescence.

In one embodiment, the derivative of NMN can be selected from among alpha nicotinamide mononucleotide (α-NMN), dihydronicotinamide mononucleotide (denoted NMN-H), the compound of formula (I):

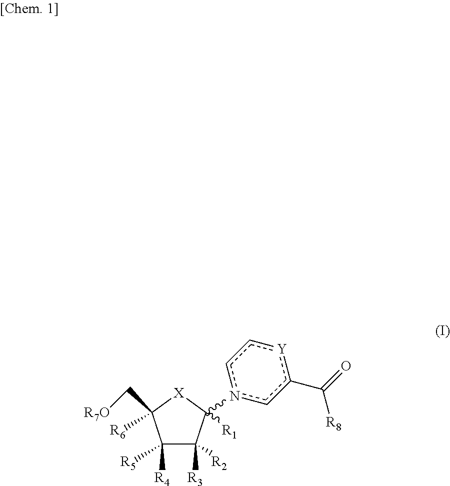

or one of the pharmaceutically acceptable stereoisomers, salts, hydrates, solvates or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ et R₅ are each independently selected from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from among a proteinogenic amino acid;
R₆ is selected from among H, azido, cyano, C₁-C₈ alkyl, C₁-C₈ thio-alkyl, C₁-C₈ heteroalkyl and OR; wherein R is selected from H and (C₁-C₈) alkyl;
R₇ is selected from among H, P(O)R9R10, P(S)R9R10 and

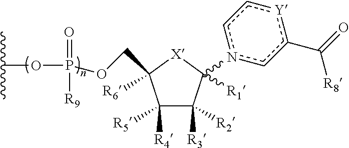

wherein n is an integer equal to 1 or 3; in which
R₉ and R₁₀ are each independently selected from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; and P(O)(OH)OP(O)(OH)₂.
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among a hydroxyl, (C₁-C₁₀) alkyl, (C₁-C₆) alkoxy, a halogen, a nitro, and a cyano; or
R₉ and R₁₀, together with the phosphorus atoms to which they are attached, form a 6-membered ring in which —R₉-R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
   R₉ and R₁₀, together with the phosphorus atoms to which they are attached, form a 6-membered ring in which —R₉-R₁₀— represents —O—CH—CH—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃ and halogen; wherein R₁₃ and R₁₄ are each independently selected from among H, (C₁-C₈) alkyl and (C₁-C₈) alkyl aryl;
Y is selected from among CH, CH₂, C(CH₃)₂ and CCH₃;
[symbol] represents a single or double bond along Y; and
[symbol] represents the alpha or beta anomer depending on the position of R₁
   or one of the stereoisomers, one of the salts, one of the hydrates, one of the solvates or one of the crystals thereof.
      or
      the compound of formula (Ia):

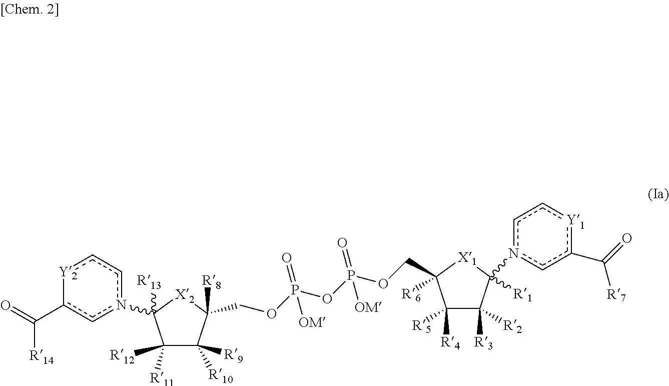

or one of the stereoisomers, one of the salts, one of the hydrates, one of the solvates or one of the crystals thereof in which:
X′₁ and X′₂ are independently selected from among O, CH₂, S, Se, CHF, CF₂, and C═CH₂;
R′₁ and R′13 are independently selected from among H, azido, cyano, a C₁-C₈ alkyl, a C₁-C₈ thio-alkyl, a C₁-C₈ heteroalkyl, and OR, wherein R is selected from H and a C₁-C₈ alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH₂ group; wherein R_{AA} is a side chain selected from a proteogenic amino acid;
R′₆ and R′₈ are independently selected from among H, an azido, a cyano, a C₁-C₈ alkyl and OR, wherein R is selected from among H and a C₁-C₈ alkyl;
R′₇ and R′₁₄ are independently selected from among H, OR, NHR, NRR′, NH—NHR, SH, CN, N₃ and a halogen, wherein R and R′ are independently selected from among H and a (C₁-C₈) alkyl aryl;
Y′₁ and Y′₂ are independently selected from among CH, CH₂, C(CH₃)₂ or CCH³;
M′ is selected from among H or a suitable counter-ion;
[symbol] represents a single or double bond depending on Y′₁ and Y′₂; and [symbol] represents an alpha or beta anomer depending on the position of R′₁ and R′₁₃;
   and combinations thereof.

In a first preferred embodiment, the pharmaceutically acceptable derivative is the compound having formula (I).

In one variant of the first embodiment, X represents un oxygen.

In one variant of the first embodiment, R₁ and R₆ are each independently a hydrogen.

In one variant of the first embodiment, R₂, R₃, R₄ and R₅ are each independently a hydrogen or OH.

In one variant of the first embodiment, Y is CH.

In one variant of the first embodiment, Y is CH₂.

In one variant of the first embodiment, R₇ is a hydrogen.

In one variant of the first embodiment, R₇ is P(O)(OH)₂.

In one variant of the first embodiment,

X represents an oxygen; and/or
R₁ and R₆ each independently represent a hydrogen; and/or
R₂, R₃, R₄ and R₅ each independently represent hydrogen, or R₂, R₃, R₄ and R₅ independently represent OH; and/or
Y represents a CH or a CH₂; and/or
R₇ represents P(O)R₉R₁₀, wherein R₉ and R₁₀ are independently selected from among OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, C₁-C₈ aryl alkyl, C₁-C₈ alkyl aryl, C₁-C₈ heteroalkyl, C₁-C₈ heterocycloalkyl, heteroaryl, and NHCR_{A}R_{A′}C(O)R₁₂.

In one particularly preferred embodiment, the compound of the invention is selected from among the compounds having the formula I-A to I-J:

TABLE 1

| Compounds | |
|---|---|
| (anomers) | Structure |
| I-A (beta) | 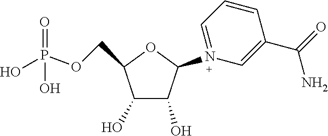 |
| I-B (alpha) | 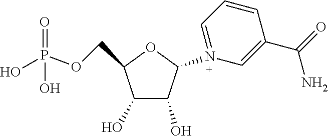 |
| I-C (beta) | 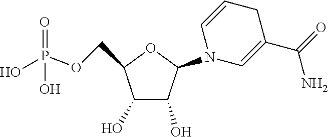 [SMILES: NC(=O)C1=CN([C@@H]2O[C@H](COP(=O)(O)O)[C@@H](O)[C@H]2O)C=CC1] |
| I-D (alpha) | 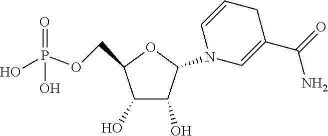 [SMILES: NC(=O)C1=CN([C@H]2O[C@H](COP(=O)(O)O)[C@@H](O)[C@H]2O)C=CC1] |
| I-E (beta) | 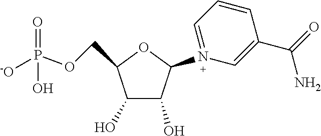 |
| I-F (alpha) | 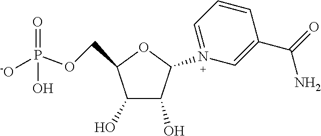 |
| I-G (beta) | 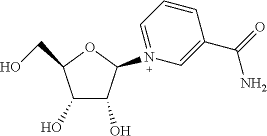 |
| I-H (alpha) | 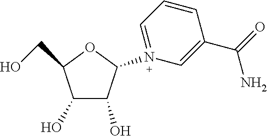 |
| I-I (beta) | 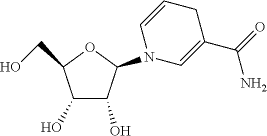 [SMILES: NC(=O)C1=CN([C@@H]2O[C@H](CO)[C@@H](O)[C@H]2O)C=CC1] |
| I-J (alpha) | 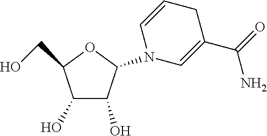 [SMILES: NC(=O)C1=CN([C@H]2O[C@H](CO)[C@@H](O)[C@H]2O)C=CC1] |

In a preferred second embodiment, the pharmaceutically acceptable derivative is the compound having the formula (Ia).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11, and R′12 each independently represent a hydrogen.

In one variant of the second embodiment, R′2, R′3, R′4, R′5, R′9, R′10, R′11, and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound according to the invention is selected from among the compounds having the formula Ia-A to Ia-I:

TABLE 2

| Compounds | |
|---|---|
| (anomers) | Structure |
| Ia-A (beta, beta) | 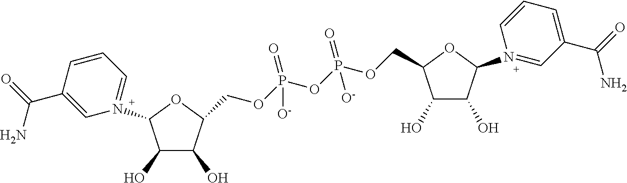 |
| Ia-B (beta, alpha) | 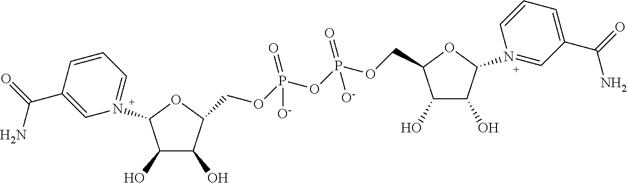 |
| Ia-C (alpha, alpha) | 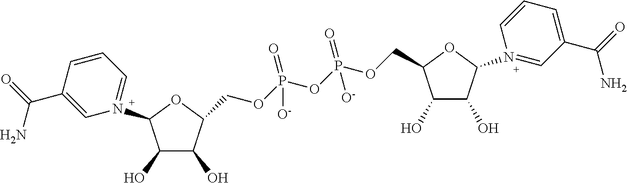 |
| Ia-D (beta, beta) | 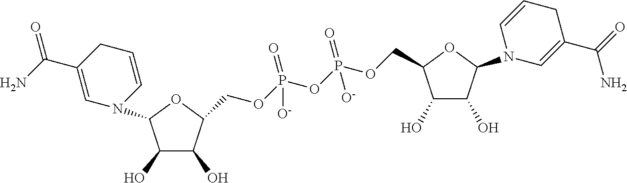 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 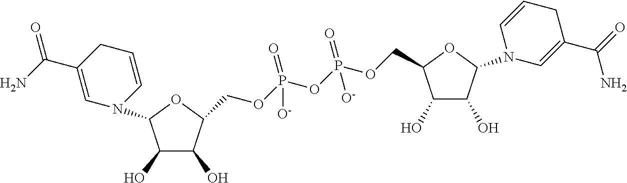 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 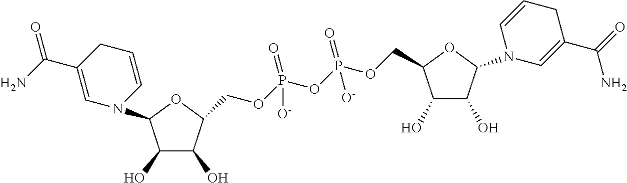 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 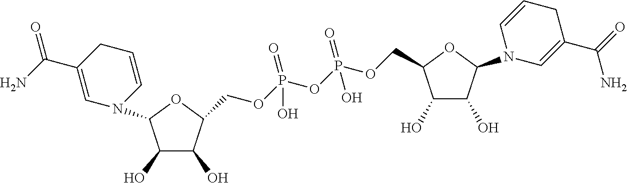 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 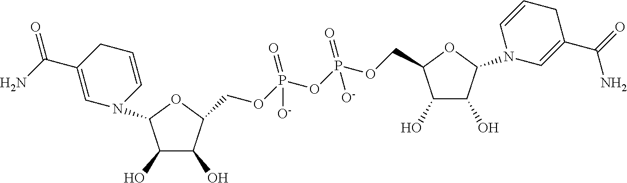 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 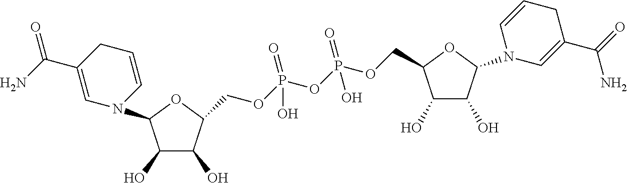 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

In one variant of the first preferred embodiment, the pharmaceutically acceptable derivative is alpha-NMN of formula:

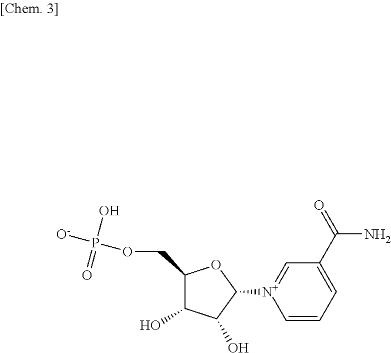 [SMILES: NC(=O)c1ccc[n+]([C@H]2O[C@H](COP(=O)([O-])O)[C@@H](O)[C@H]2O)c1]

In one preferred embodiment, the pharmaceutically acceptable derivative is NMN-H:

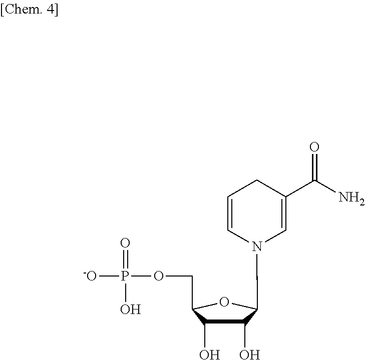

Advantageously, the pharmaceutically acceptable precursor is nicotinamide riboside (denoted NR):

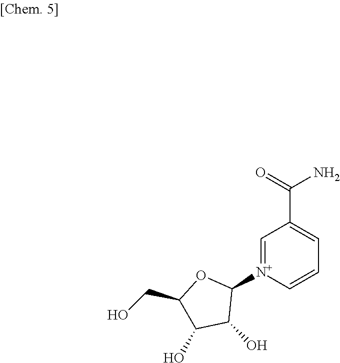 [SMILES: NC(=O)c1ccc[n+]([C@@H]2O[C@H](CO)[C@@H](O)[C@H]2O)c1]

or dihydronicotinamide riboside (denoted —NR—H) having the formula:

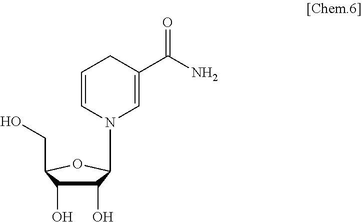

Advantageously, the nicotinamide mononucleotide (NMN), a pharmaceutically acceptable precursor thereof, a pharmaceutically acceptable derivative thereof, or a pharmaceutically acceptable salt thereof, can be administered via oral, ocular, sublingual, parenteral, transcutaneous, vaginal, peridural, intravesical, rectal or inhalation route.

Preferably, the nicotinamide mononucleotide (NMN), a pharmaceutically acceptable precursor thereof, a pharmaceutically acceptable derivative thereof or a pharmaceutically acceptable salt thereof, can be administered via oral or parenteral route, preferably via oral route.

Preferably, the parenteral route can be selected from among the intra-arterial route, intravenous route, intramuscular route, subcutaneous route, intraperitoneal route, further preferably the intravenous route.

Advantageously, the nicotinamide mononucleotide (NMN), a pharmaceutically acceptable precursor thereof, a pharmaceutically acceptable derivative thereof or a pharmaceutically acceptable salt thereof, can be in the form of a tablet, hard capsule, sachet, granule, soft shell capsule, lozenge, lyophilizate, suspension, gel, syrup, solution, water/oil emulsion, oil/water emulsion, oil, cream, milk, spray, ointment, phial, suppository, eye drops, vaginal ovula, vaginal capsule, liquid for inhalation, dry powder inhalator, inhalator with pressurized metering valve, a powder.

Preferably, the nicotinamide mononucleotide (NMN), a pharmaceutically acceptable precursor thereof, a pharmaceutically acceptable derivative thereof, or a pharmaceutically acceptable salt thereof, is in the form of a powder, suspension, solution, water/oil emulsion or oil/water emulsion.

Advantageously, the NMN, a pharmaceutically acceptable precursor thereof, a pharmaceutically acceptable derivative thereof, or a pharmaceutically acceptable salt thereof, can be used in an amount of between 0.01 mg/kg/day and 500 mg/kg/day, preferably between 0.1 mg/kg/day and 350 mg/kg/day, more preferably between 0.5 and 100 mg/kg/day, further preferably from 5 mg/kg/day to 50 mg/kg/day.

Advantageously, the NMN, a pharmaceutically acceptable precursor thereof, a pharmaceutically acceptable derivative thereof, or a pharmaceutically acceptable salt thereof, can be administered to a mammal, preferably a human.

In one embodiment, the NMN, a pharmaceutically acceptable precursor thereof, a pharmaceutically acceptable derivative thereof, or a pharmaceutically acceptable salt thereof, can be administered to a human of at least 45 years of age, preferably at least 50 years of age, preferably at least 55 years of age, preferably at least 60 years of age, preferably at least 65 years of age, more preferably at least 70 years of age, further preferably at least 75 years of age for treatment of immunosenescence.

In one embodiment, the NMN, a pharmaceutically acceptable precursor thereof, a pharmaceutically acceptable derivative thereof, or a pharmaceutically acceptable salt thereof, can be administered to a human of at least 15 years of age, preferably at least 20 years of age, more preferably at least 25 years of age, further preferably at least 30 years of age, still further preferably at least 35 years of age for the prevention of immunosenescence.

Advantageously, the NMN, a pharmaceutically acceptable precursor thereof, a pharmaceutically acceptable derivative thereof, or a pharmaceutically acceptable salt thereof, can be administered in combination with at least one other therapeutic agent.

In at least one embodiment, the at least one other therapeutic agent is a vaccine that can be selected from among attenuated live vaccines, inactivated vaccines, multivalent vaccines, or combination vaccines.

In at least one embodiment, said vaccine is selected from among a vaccine against a virus, a bacterium, a parasite or yeast and/or fungus, or combinations thereof.

Preferably, said vaccine is a vaccine against a virus selected from among Influenzavirus, Coronavirus, Respirovirus, Pneumovirus, Metapneumovirus, Adenovirus, Enterovirus, Rhinovirus, Hepatovirus, Erbovirus, Aphtovirus, Norovirus, Alphavirus, Rubivirus, Flavivirus, Hepacivirus, Pestivirus, Ebola, Morbillivirus, Rubulavirus, Henipavirus, Arenavirus, Orthobunyavirus, Phlebovirus, Rotavirus, Simplexvirus, Varicellovirus, Papillomavirus, Cytomegalovirus or combinations thereof.

More preferably, the vaccine against the virus is selected from among Influenzavirus, Coronavirus, Rhinovirus, Respirovirus, Pneumovirus or Metapneumovirus, further preferably Influenzavirus.

In one embodiment, said vaccine is a vaccine against a bacterium selected from among Pneumococcus, Streptococcus, Corynebacterium, Clostridium, Mycobacterium, Bordetella, Neisseria and combinations thereof.

Preferably, the Mycobacterium is Mycobacterium tuberculosis. Preferably, said Neisseria is Neisseria meningitis.

In one embodiment, said vaccine is a vaccine against a parasite selected from among Schistosoma, Leishmania, Babesia and combinations thereof.

In one embodiment, said vaccine is a vaccine against a yeast and/or fungus selected from among Trichophyton, Toxoplasma, Eimeria, Candida and combinations thereof.

In one embodiment, the at least one therapeutic agent is a radiotherapy, chemotherapy, or combinations thereof.

Advantageously, radiotherapy is selected from among X- or gamma-irradiation.

Advantageously, chemotherapy can be selected from among an antimetabolite, an alkylating agent, a topoisomerase inhibitor, an anthracycline, and combinations thereof.

Advantageously, the alkylating agent can be selected from among dacarbazine, temozolomide, streptozocin, cyclophosphamide, ifosfamide, melphalan, procarbazine, busulfan, triphosphoramide, hexamethylmelamine, chlormethine, platinum salts such as cisplatin, carboplatin, oxaliplatin and combinations thereof.

Advantageously, the at least one therapeutic agent is an immunosuppressive treatment.

Advantageously, the immunosuppressive treatment can be selected from among an antimetabolite, TNF alpha inhibitor, interleukin-1 (IL1) inhibitor, a derivative of cortisone, inhibitor of calcineurin, rapamycin, an anti-CD25 antibody, lymphoablative treatment and combinations thereof.

Advantageously, the antimetabolite can be selected from among azathioprine, methotrexate, mycophenolic acid, mycophenolate mofetil, fludarabine, and combinations thereof.

Advantageously, the derivative of cortisone is selected from among betamethasone, ciprofloxacin, cortivazol, dexamethasone, fludrocortisone, methylprednisolone, prednisolone, triamcinolone, and combinations thereof.

Advantageously, the inhibitor of calcineurin can be selected from among cyclosporine, tacrolimus, and combinations thereof.

Advantageously, the lymphoablative treatment can be selected from among chemotherapy, radiotherapy, an alkylating agent, an intercalator, an anthracycline, anti-thymocyte globulins, CD52 monoclonal antibody, OKT3 monoclonal antibody, and combinations thereof.

Advantageously, the NMN, a pharmaceutically acceptable precursor thereof, a pharmaceutically acceptable derivative thereof, or a pharmaceutically acceptable salt thereof, can be administered concomitantly with the administering of at least one additional therapeutic agent.

Advantageously, the NMN, a pharmaceutically acceptable precursor thereof, a pharmaceutically acceptable derivative thereof, or a pharmaceutically acceptable salt thereof, can be administered for a time of between 10 and 60 days, preferably between 14 and 42 days, more preferably for about 30 days preceding the administering of at least one additional therapeutic agent.

Advantageously, the immunodeficiency can be primary immunodeficiency or secondary immunodeficiency.

Advantageously, primary immunodeficiency is of hereditary origin and can be selected from among primary predominantly humoral immunodeficiency, primary predominantly cellular immunodeficiency, combined deficiency, deficiency of complement proteins, or deficiency of macrophage and polynuclear cell activity.

Advantageously, primary predominantly humoral immunodeficiency can be selected from among sex-linked or Bruton disease-related agammaglobulinemia, common variable hypogammaglobulinemia or a selective deficiency of immunoglobulins such as a deficiency of IgA, IgD, IgG and/or IgM.

Advantageously, primary predominantly cellular immunodeficiency can be selected from among 22q11 microdeletion or Di George syndrome, Hong and Good syndrome, Nezelof syndrome, purine nucleoside phosphorylase deficiency, isolated T-lymphocyte deficiency.

Advantageously, the combined deficiency can be selected from among severe combined immunodeficiency through deficiency of adenosine deaminase, bare-lymphocyte syndrome, congenital amegakaryocytic thrombocytopenia with abnormal development of T and B lines, Wiskott-Aldrich syndrome, ataxia telangiectasia, chronic mucocutaneous candidiasis, acrodermatitis enteropathica, Hyper IgE syndrome.

Advantageously, deficiency of complement proteins is selected from among deficiency of complement component 1 (C1) inhibitor (hereditary Quincke's oedema), C3 deficiency, C4 deficiency, C5, C6, C7, C8 and/or C9 deficiency.

Advantageously, deficiency of macrophage or polynuclear cell activity can be selected from among septic granulomatosis, myeloperoxidase deficiency, Chediak Higashi syndrome, actin dysfunction, Shwachman syndrome.

In one embodiment, immunodeficiency can be secondary.

Advantageously, secondary immunodeficiency can be due to HIV infection, sarcoidosis, a thymoma, thymic hypoplasia, acute leukaemia, chronic lymphoid leukaemia, malignant lymphoma, multiple myeloma, Waldenström's disease, treatment with a cortisone derivative, immunosuppressive treatment, thymus ablation, chemotherapy, radiotherapy, a viral infection, a bacterial infection, an infection caused by a fungus and/or yeast, an infection caused by a parasite, dietary deficiency, an auto-immune disease, chronic kidney disease, hematologic malignancy, toxic blood disease, asplenia, hyposplenia, a metabolic disease, a cancer affecting the immune system, a drug therapy, a splenectomy, a substance addiction, alcoholism, immunosenescence and combinations thereof, preferably immunosenescence.

Advantageously, the metabolic disease can be selected from among type-2 diabetes, cirrhosis, non-alcoholic hepatic steatosis, obesity, and combinations thereof.

In one embodiment, the treatment can be immunosuppressive treatment, a corticoid, chemotherapy, radiotherapy, and combinations thereof.

Advantageously, the immunodeficiency can be characterized by at least one of the markers selected from among neutropenia, lymphopenia, a CD4/CD8 ratio lower than 1, and combinations thereof.

Preferably, the invention is particularly useful for treating or preventing immunosenescence.

Advantageously, the NMN, a pharmaceutically acceptable precursor thereof, a pharmaceutically acceptable derivative thereof, or a pharmaceutically acceptable salt thereof, can be administered to a human of at least 40 years of age, preferably at least 45 years of age, preferably at least 50 years, of age, preferably at least 55 years of age, preferably at least 60 years of age, preferably at least 65 years of age, more preferably at least 70 years of age, further preferably at least 75 years of age, still further preferably at least 80 years of age for the treatment of immunosenescence.

Advantageously, the NMN, a pharmaceutically acceptable precursor thereof, a pharmaceutically acceptable derivative thereof, or a pharmaceutically acceptable salt thereof, and the compositions comprising the same can be administered to a human of at least 15 years of age, preferably at least 20 years of age, more preferably at least 25 years of age, further preferably at least 30 years of age, still further preferably at least 35 years of age for the prevention of immunosenescence.

Advantageously, the decrease in immunosenescence can be measured by the reduction of a marker selected from among thymic involution, at least one cytokine selected from among IL1, IL2, IL6, IL12, I15, IL18, IL22; TNF alpha, Interferon gamma, C-reactive protein, the number of resident senescent T cells in the spleen, the level of circulating IgG immunoglobulin produced by memory B cells, the level of circulating IgA immunoglobulin produced by memory B cells, and combinations thereof.

Advantageously, the decrease in immunosenescence can be measured by an increase in a marker selected from among the production of new naive T cells, the capacity to respond to new antigens, the accumulation of memory T cells, the number of circulating B cells, the levels of circulating immunoglobulins produced by naive cells (IgD and/or IgM), vaccinal immunogenicity, an increase in the CD4/CD8 ratio, IL1-Ralpha level, IL4 level, IL10 level, TGF-beta 1 level, cell sedimentation rate, and combinations thereof.

A further subject of the invention is a composition comprising nicotinamide mononucleotide, a pharmaceutically acceptable precursor thereof, a pharmaceutically acceptable derivative thereof, or a pharmaceutically acceptable salt thereof, and at least one pharmaceutically acceptable excipient for the prevention and/or treatment of immunodeficiency, preferably immunosenescence, such as defined above.

### Definitions

In the present invention, the following terms have the following meanings.

Unless otherwise indicated, the nomenclature of substituents which are not explicitly defined in the present invention is obtained by naming the terminal portion of the functional group followed by the adjacent functional group towards the point of attachment.

“Alkyl” on its own or as part of another substituent refers to a hydrocarbyl radical having the formula CnH2n+1 in which n is a number greater than or equal to 1. In general, the alkyl groups of this invention include from 1 to 12 carbon atoms, preferably from 1 to 8 carbon atoms, more preferably from 1 to 6 carbon atoms, even more preferably from 1 to 2 carbon atoms. The alkyl groups may be linear or branched and may be substituted as indicated in the present invention. The alkyls that are suitable for the purposes of implementation of the invention may be selected from among methyl, ethyl, n-propyl, i-propyl, n-butyl, i-butyl, s-butyl and t-butyl; pentyl and its isomers such as n-pentyl and iso-pentyl; and hexyl and its isomers such as n-hexyl and iso-hexyl; heptyl and its isomers (for example n-heptyl, iso-heptyl); octyl and isomers thereof (for example n-octyl, iso-octyl); nonyl and isomers thereof (for example n-nonyl, iso-nonyl); decyl and its isomers (for example n-decyl, iso-decyl); undecyl and its isomers; dodecyl and its isomers. Preferably, the alkyl groups may be selected from among methyl, ethyl, n-propyl, i-propyl, n-butyl, i-butyl, s-butyl, t-butyl, n-pentyl, n-hexyl, n-heptyl, n-octyl, n-nonyl, and n-decyl. The saturated and branched alkyl groups may be selected, without limitation, from among isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, and 3,3-diethylhexyl. The preferred alkyl groups are the following: methyl, ethyl, n-propyl, i-propyl, n-butyl, i-butyl, s-butyl and t-butyl. Cx-Cy-alkyls refer to alkyl groups that comprise from x to y carbon atoms.

When the suffix “ene” (“alkylene”) is used in conjunction with an alkyl group, it indicates that the alkyl group as defined herein has two single bonds as points of attachment to other groups. The term “alkylene” includes methylene, ethylene, methylmethylene, propylene, ethylethylene, and 1,2-dimethylethylene.

The term “alkenyl” as used herein refers to an unsaturated hydrocarbyl group, which may be linear or branched, that comprises one or more carbon-carbon double bonds. The alkenyl groups that are suitable comprise between 2 and 12 carbon atoms, preferably between 2 and 8 carbon atoms, and even more preferably between 2 and 6 carbon atoms. Examples of alkenyl groups are ethenyl, 2-propenyl, 2-butenyl, 3-butenyl, 2-pentenyl and isomers thereof, 2-hexenyl and its isomers, 2,4-pentadienyl and other similar groups.

The term “alkynyl”, as used herein, refers to a class of monovalent unsaturated hydrocarbyl groups, in which the unsaturation results from the presence of one or more carbon-carbon triple bond(s). The alkynyl groups generally, and preferably, have the same number of carbon atoms as described here above for the alkenyl groups. Without limitation, some examples of alkynyl groups include ethynyl, 2-propynyl, 2-butynyl, 3-butynyl, 2-pentynyl and isomers thereof, 2-hexynyl and the isomers thereof, etc.

«Alkoxy» refers to an alkyl group as defined here above, which is attached to another moiety by means of an oxygen atom. Examples of alkoxy groups include the groups: methoxy, isopropoxy, ethoxy, tert-butoxy, and the like. The alkoxy groups may be optionally substituted by one or more substituent(s). The alkoxy groups included in the compounds of this invention may be optionally substituted with a solubilizing group.

«Aryl», as used herein, refers to a polyunsaturated aromatic hydrocarbyl group having a single ring (for example phenyl) or multiple aromatic rings that are fused together (for example naphthyl) or covalently bonded, which generally contains 5 to 18 atoms, preferably 5 to 12, on a more preferred basis 6 to 10, with at least one of the said rings being aromatic. The aromatic ring may optionally include one or two additional rings (cycloalkyl, heterocyclyl, or heteroaryl) fused thereto. The aryl is also intended to include partially hydrogenated derivatives of the carbocyclic systems listed herein. Examples of aryl include phenyl, biphenylyl, biphenylenyl, 5- or 6-tetralinyl; naphthalene-1- or -2-yl; 4-, 5-, 6 or 7-indenyl; 1-, 2-, 3-, 4-, or 5-acenaphthylenyl; 3-, 4-, or 5-acenaphthenyl; 1-, or 2-pentalenyl; 4-, or 5-indanyl; 5-, 6-, 7-, or 8-tetrahydronaphthyl; 1,2,3,4-tetrahydronaphthyl; 1,4-dihydronaphthyl; 1-, 2-, 3-, 4-, or 5-pyrenyl.

When at least one carbon atom in an aryl group is replaced by a heteroatom, the resulting ring is referred to herein as a “heteroaryl” ring.

«Alkylaryl» designates an aryl group substituted by an alkyl group.

“Amino acid” refers to an alpha-amino carboxylic acid, that is to say, a molecule comprising a carboxylic acid functional group and an amino functional group in the alpha position of the carboxylic acid group, for example a proteinogenic amino acid or a non-proteinogenic amino acid.

“Proteinogenic amino acid” refers to an amino acid that is incorporated into the proteins during the translation of the messenger RNA by the ribosomes in living organisms, that is to say, Alanine (ALA), Arginine (ARG), Asparagine (ASN), Aspartate (ASP), Cysteine (CYS), Glutamate (glutamic acid) (GLU), Glutamine (GLN), Glycine (GLY), Histidine (HIS), Isoleucine (ILE), Leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophan (TRP), Tyrosine (TYR), or Valine (VAL).

“Non-proteinogenic amino acid” as used herein refers to an amino acid that is not naturally encoded or found in the genetic code of a living organism. Without limitation, some examples of non-proteinogenic amino acid are: ornithine, citrulline, argininosuccinate, homoserine, homocysteine, cysteine-sulfinic acid, 2-aminomuconic acid, δ-aminolevulinic acid, β-alanine, cystathionine, γ-aminobutyrate, dihydroxyphenylalanine (DOPA), 5-hydroxytryptophan, D-serine, ibotenic acid, α-aminobutyrate, 2-aminoisobutyrate, D-leucine, D-valine, D-alanine, and D-glutamate.

The term “cycloalkyl” as used herein refers to a cyclic alkyl group, i.e. a monovalent, saturated or unsaturated hydrocarbyl group, having 1 or 2 ring structures. The term “cycloalkyl” includes monocyclic or bicyclic hydrocarbyl groups. The cycloalkyl groups may comprise 3 or more carbon atom(s) in the ring and generally, according to the present invention, comprise from 3 to 10, more preferably from 3 to 8 carbon atoms, and even more preferably from 3 to 6 carbon atoms. Examples of cycloalkyl groups include, but are not limited to, cyclopropyl, cyclobutyl, cyclopentyl, cyclohexyl, with cyclopropyl being particularly preferred.

The term “pharmaceutically acceptable excipient” refers to an inert carrier or support substance used as a solvent or diluent within which the active ingredient is formulated and/or administered, and which does not produce an adverse, allergic or other reaction when it is administered to an animal, preferably to a human. This includes all solvents, dispersing media, coatings, antibacterial and antifungal agents, isotonic agents, absorption retardants, and other similar ingredients. For human administering, the preparations must meet specific standards of sterility, general safety and purity, as required by the regulatory authorities, such as for example the Food and Drug Administering (FDA) in the United States of America, or the European Medicines Agency (EMA). Within the meaning of the invention, “pharmaceutically acceptable excipient” includes all pharmaceutically acceptable excipients as well as all pharmaceutically acceptable carriers, diluents and/or adjuvants.

“White blood cells” or “leukocytes” are cells of the immune system. This generic term includes neutrophils, eosinophils, basophils, T and B lymphocytes and monocytes.

“Halogen” or “halo” refers to fluoro, chloro, bromo or iodo. The preferred halo groups are fluoro and chloro.

“Haloalkyl” alone or in combination, refers to an alkyl radical having the meaning as defined here above, in which one or more hydrogen atom(s) are replaced by a halogen as defined here above. By way of examples of such haloalkyl radicals, the following may be cited: chloromethyl, 1-bromoethyl, fluoromethyl, difluoromethyl, trifluoromethyl, 1,1,1-trifluoroethyl, and similar radicals. ‘Cx-Cy-haloalkyl’ and ‘Cx-Cy-alkyl’ refer to alkyl groups that contain from x to y carbon atoms. The preferred haloalkyl groups are difluoromethyl and trifluoromethyl.

“Heteroalkyl” refers to an alkyl group as defined here above, in which one or more carbon atom(s) are replaced by a heteroatom selected from among oxygen, nitrogen and sulfur atoms. In the heteroalkyl groups, the heteroatoms are bonded along the alkyl chain only to carbon atoms, that is to say, each heteroatom is separated from every other heteroatom by at least one carbon atom. However, the nitrogen and sulfur heteroatoms may optionally be oxidised and the nitrogen heteroatoms may optionally be quaternised. A heteroalkyl is bonded to another group or molecule only by means of a carbon atom, that is to say, the bonding atom is not selected from the heteroatoms included in the heteroalkyl group.

The term “heteroaryl” as used herein, alone or as part of another group, refers to, but is not limited to, aromatic rings of 5 to 12 carbon atoms or ring systems containing 1 or 2 rings that are fused or covalently bonded, and generally containing 5 or 6 atoms, with at least one of the said rings being aromatic; in which one or more carbon atom(s) in one or more of these rings are replaced by oxygen, nitrogen and/or sulfur atoms, it being possible for the nitrogen and sulfur heteroatoms to optionally be oxidised and for the nitrogen heteroatoms to optionally be quaternised. These rings may be fused to an aryl, cycloalkyl, heteroaryl or heterocyclyl ring. Without limitation, some examples of such heteroaryls include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, dioxinyl, thiazinyl, triazinyl, imidazo [2,1-b] [1,3] thiazolyl, thieno [3,2-b] furanyl, thieno [3,2-b] thiophenyl, thieno [2,3-d] [1,3] thiazolyl, thieno [2,3-d] imidazolyl, tetrazolo [1,5-a] pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1, 2,3-benzothiadiazolyl, 2,1,3-benzothiadiazolyl, thienopyridinyl, purinyl, imidazo [1,2-a] pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

When at least one carbon atom in a cycloalkyl group is replaced by a heteroatom, the resulting ring is referred to herein as “heterocycloalkyl” or “heterocyclyl”.

The terms “heterocyclyl”, “heterocycloalkyl”, or “heterocyclo”, as used herein by themselves or as part of another group, refer to non-aromatic cyclic groups, either fully saturated or partially unsaturated (for example, 3 to 7 membered monocyclic, 7 to 11 membered bicyclic groups or containing a total of 3 to 10 ring atoms), which have at least one heteroatom in at least one ring containing a carbon atom. Each ring of the heterocyclic group containing a heteroatom may have 1, 2, 3, or 4 heteroatoms selected from among nitrogen, oxygen and/or sulfur atoms, where the nitrogen and sulfur heteroatoms may optionally be oxidised, and the nitrogen heteroatoms may optionally be quaternised. Any whichever of the carbon atoms of the heterocyclic group may be substituted by an oxo (for example piperidone, pyrrolidinone). The heterocyclic group may be attached to any heteroatom or carbon atom in the ring or ring system, where the valence so permits. The rings of multi-ring heterocycles may be fused, bridged and/or connected by one or more spiro atoms. Exemplary heterocyclic groups include, but are not limited to, the following groups: oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholine-4-yl, thiomorpholine-4-ylsulf oxide, thiomorpholine-4-ylsulfone, 1,3-dioxolanyl, 1,4-oxathianyl, 1H-pyrrolizinyl, tetrahydro-1,1-dioxothiophenyl, N-formylpiperazinyl, and morpholine-4-yl.

“Leukopenia” is a blood disorder characterized by a white blood cell count of less than 4,000 cells/μL of blood.

“Lymphopenia” is a blood disorder in which the number of lymphocytes on a blood count is less than normal, i.e., less than 1500 per mm³.

The term “neutropenia” is a haematological disorder characterized by a low level of granulocytes, or polynuclear cells, neutrophils in the blood. Normal neutropenia is defined as less than 2000 neutrophils/μL of blood. Mild neutropenia is defined as 1,000 to 1,500 neutrophils/μL of blood. Moderate neutropenia is 500 to 1,000 neutrophils/μL of blood. Severe neutropenia is defined as less than 500 neutrophils/μL of blood. Agranulocytosis is defined as a neutrophil count of less than 100/mm3 of blood.

“Elderly” means a human selected from a human at least 60 years of age, a human at least 65 years of age, a human at least 70 years of age, a human at least 75 years of age, a human at least 80 years of age, a human at least 85 years of age, a human at least 90 years of age, a human at least 95 years of age.

The term “precursor” as used herein also refers to pharmacologically acceptable derivatives of compounds having the formula (I) or (Ia) such as esters, of which the in vivo biotransformation product is the active drug. Precursors are characterised by increased bioavailability and are readily metabolised into active compounds in vivo. The precursors that are appropriate for the purposes of the invention include in particular carboxylic esters, in particular alkyl esters, aryl esters, acyloxyalkyl esters, and the carboxylic esters of dioxolene; ascorbic acid esters.

The term “pharmaceutically acceptable” refers to the state of being approved, or with the likelihood of being potentially approved by a regulatory body or listed in a recognised pharmacopoeia for use in animals, and more preferably in humans. It may pertain to a substance that is not biologically or otherwise undesirable; that is to say, the substance may be administered to an individual without causing adverse biological effects or deleterious interactions with one of the components of the composition within which it is contained. Preferably, a “pharmaceutically acceptable” salt or excipient refers to any salt or any excipient that is authorised by the European Pharmacopoeia (denoted as “Ph. Eur.”) and the American Pharmacopoeia (generally referred to as “United States Pharmacopeia (USP)».

The term “active ingredient” refers to a molecule or a substance which when administered to a subject slows down or stops the progression, aggravation or deterioration of one or more symptom(s) of a disease or a condition; relieves the symptoms of a disease or a condition; cures a disease or a condition. According to one of these embodiments, the therapeutic ingredient is a small molecule, which is natural or synthetic. According to another embodiment, the therapeutic ingredient is a biological molecule such as, for example, an oligonucleotide, a small interfering RNA (siRNA), a microRNA (miRNA), a DNA fragment, an aptamer, an antibody and the like. “Pharmaceutically acceptable salts” include the acid addition salts and base addition salts of these said salts. Suitable acid addition salts are formed from acids that form non-toxic salts. Examples that may be cited include: acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, and salts of xinofoate. Suitable basic salts are formed from bases which form non-toxic salts. By way of examples, mention may be made of the salts of: aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine, and zinc. Hemisalts of acids and bases may also be formed, for example, hemisulfates and salts of chemical calcium. The preferred pharmaceutically acceptable salts are hydrochloride/chloride, bromide/hydrobromide, bisulfate/sulfate, nitrate, citrate and acetate.

Pharmaceutically acceptable salts can be prepared by one or more of the following methods:
i. by reacting the compound with the desired acid;
ii. by reacting the compound with the desired base;
iii. by removing an acid or base labile protecting group under basic or acidic conditions from a suitable precursor of the compound, or by ring opening of a suitable cyclic precursor, for example a lactone or a lactam, using the desired acid; or
iv. by converting one salt of the compound into another by reacting the initial salt with an appropriate acid or by means of an appropriate ion exchange column.

All of these reactions are generally carried out in solution. The salt can precipitate out of the solution and may be collected by filtration or may be recovered by evaporation of the solvent. The degree of ionisation of the salt may vary from completely ionised to almost non-ionised.

The term “Solvate” is used herein to describe a molecular complex that comprises the compound of the invention and one or more pharmaceutically acceptable solvent molecules, for example ethanol.

The term “substituent” or “substituted” indicates that a hydrogen radical on a compound or a group is replaced by any desired group which is substantially stable under the reaction conditions in an unprotected form or when it is protected by a protecting group. Examples of preferred substituents include, but are not limited to: a halogen (chloro, iodo, bromo, or fluoro); an alkyl; an alkenyl; an alkynyl, as described here above; a hydroxy; an alkoxy; a nitro; a thiol; a thioether; an imine; a cyano; an amido; a phosphonato; a phosphine; a carboxyl; a thiocarbonyl; a sulfonyl; a sulfonamide; a ketone; an aldehyde; an ester; an oxygen (—O); a haloalkyl (for example, trifluoromethyl); a cycloalkyl, which may be condensed-ring or non-condensed-ring monocyclic or polycyclic (for example, cyclopropyl, cyclobutyl, cyclopentyl or cyclohexyl); or a heterocycloalkyl, which may be condensed-ring or non-condensed-ring monocyclic or polycyclic (for example, pyrrolidinyl, piperidinyl, piperazinyl, morpholinyl, or thiazinyl); fused or unfused monocyclic or polycyclic, aryl or heteroaryl (for example, aryl, heteroaryl, pyrrolidinyl, piperidinyl, piperazinyl, morpholinyl, or thiazinyl); fused or unfused monocyclic or polycyclic (for example, aryl, heteroaryl, pyrrolidinyl, piperidinyl, piperazinyl, morpholinyl, or thiazinyl), phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazidinyl, pyridaziminyl, pyridaziminyl, benzimidazolyl, benzothiophenyl, or benzofuranyl); amino (primary, secondary or tertiary); CO₂CH₃; CONH2; OCH₂CONH₂; NH₂; SO₂NH₂; OCHF₂; FC₃; OCF₃; moreover these groups may also be optionally substituted by a fused ring bridge or structure, for example —OCH₂O—. These substituents may optionally be further substituted by a substituent selected from among these groups. In certain representations, the term “substituent” or the adjective “substituted” refers to a substituent selected from the group constituted of: an alkyl, an alkenyl, an alkynyl, a cycloalkyl, a cycloalkenyl, a heterocycloalkyl, an aryl, a heteroaryl, an arylalkyl, a heteroarylalkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR13, —NR₁₃S(O)rR₁₄, —OS(O)rR₁₄, S(O)rNR₁₁R₁₂, —O, —S, and —NR₁₃, where r is 1 or 2; R₁₁ and R₁₂, for each occurrence, are independently H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₁ and R₁₂ taken together with the nitrogen to which they are attached are an optionally substituted heterocycloalkyl, or an optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In some embodiments; the term “substituent” or the adjective “substituted” refers to a solubilising group.

The term “administering”, or a variant of this term (for example, “to administer”), refers to providing of the active ingredient, whether alone or as part of a pharmaceutically acceptable composition, to the patient who is to receive the same in the context of treatment or prevention of a condition, a symptom, or a disease.

The terms “treating”, “curing”, and “treatment”, as used herein, are meant to include the relieving, alleviation, or ablation of a condition, or a disease and/or the symptoms associated therewith.

The terms “prevent”, “impede” and “prevention”, as used in the present invention, refer to a method that serves the purpose of: delaying, or impeding or preventing the onset of a condition, or a disease and/or the symptoms associated therewith; preventing a patient from contracting a condition or a disease; or reducing the risk of a patient's contracting of a given disease or condition.

The bonds of an asymmetric carbon may be represented herein using a solid triangle ( [symbol] ), a dotted triangle ( [symbol] ) or a zigzag line ( [symbol] ).

### DETAILED DESCRIPTION OF THE INVENTION

The subject of the present invention is nicotinamide mononucleotide (NMN), a pharmaceutically acceptable derivative thereof, or a pharmaceutically acceptable salt thereof for use thereof in the prevention and/or treatment of immunodeficiency, preferably immunosenescence.

A further subject of the invention is a composition comprising NMN, a pharmaceutically acceptable derivative thereof, or a pharmaceutically acceptable salt thereof, and at least one pharmaceutically acceptable excipient for use thereof in the prevention and/or treatment of immunodeficiency, preferably immunosenescence.

Nicotinamide adenine dinucleotide (NAD) is a coenzyme present in all living cells. NAD exists in the cell either in its oxidised form NAD+, or in its reduced form NADH. The role of NAD is that of an electron carrier that is involved in the oxidation-reduction reactions of metabolism. NAD is moreover also involved in a number of cellular processes such as adenosine diphosphate (ADP) ribosylation in the context of post-translational modifications of proteins.

NAD can be synthesised de novo by the cell from amino acids such as tryptophan or aspartate.

However, such synthesis is marginal because the main pathway for NAD synthesis is the salvage pathway, by means of which the cell, and primarily the cell nucleus, recycles compounds in order to reform NAD from precursors. The precursors of NAD include niacin, nicotinamide riboside, nicotinamide mononucleotide, and nicotinamide.

NMN is one of the compounds that enable the synthesis of NAD by the salvage pathway and has the formula:

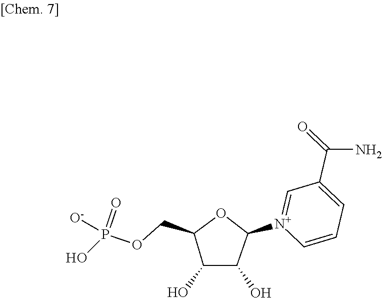 [SMILES: NC(=O)c1ccc[n+]([C@@H]2O[C@H](COP(=O)([O-])O)[C@@H](O)[C@H]2O)c1]

The inventors have effectively discovered that the use of NMN allows a decrease in immunodeficiency and particularly in immunosenescence, or at least a reduction in the signs thereof.

In one preferred embodiment, the NMN is in the form of a zwitterion. By «zwitterion» it is meant a molecular chemical species having opposed electrical charges and generally located on non-adjacent atoms of the molecule.

### Use

NMN, a pharmaceutically acceptable precursor thereof, a pharmaceutically acceptable derivative thereof, or a pharmaceutically acceptable salt thereof and compositions comprising the same can be used to treat or prevent immunodeficiency, preferably immunosenescence.

NMN, a pharmaceutically acceptable precursor thereof, a pharmaceutically acceptable derivative thereof, or a pharmaceutically acceptable salt thereof can be administered to a mammal, preferably a human.

Advantageously, immunodeficiency can be primary immunodeficiency or secondary immunodeficiency.

Primary immunodeficiency is of hereditary origin and can be selected from among predominantly humoral deficiency, predominantly cellular deficiency, combined deficiency, complement protein deficiency, or deficiency of macrophage or polynuclear cell activity.

Primary predominantly humoral deficiency can be selected from among sex-linked or Bruton's disease-related agammaglobulinemia, common variable hypogammaglobulinemia, or selective immunoglobulin deficiency such as deficiency of IgA, IgD, IgG and/or IgM.

Primary predominantly cellular immunodeficiency can be selected from among 22q11 microdeletion or Di George syndrome, Hong and Good syndrome, Nezelof syndrome, purine nucleoside phosphorylase deficiency, isolated T-lymphocyte deficiency.

Combined deficiency can be selected from among severe combined immunodeficiency through deficiency of adenosine deaminase, bare-lymphocyte syndrome, congenital amegakaryocytic thrombocytopenia with abnormal development of T and B lines, Wiskott-Aldrich syndrome, ataxia telangiectasia, chronic mucocutaneous candidiasis, acrodermatitis enteropathica, Hyper IgE syndrome. Deficiency of macrophage or polynuclear cell activity can be selected from among septic granulomatosis, myeloperoxidase deficiency, Chediak Higashi syndrome, actin dysfunction, Shwachman syndrome.

Deficiency of complement proteins can be selected from among deficiency of complement component 1 (C1) inhibitor (hereditary Quincke's oedema), C3 deficiency, C4 deficiency, C5, C6, C7, C8 and/or C9 deficiency.

Immunodeficiency can be secondary or acquired. In particular, secondary immunodeficiency can be due to HIV infection, sarcoidosis, a thymoma, thymic hypoplasia, acute leukaemia, chronic lymphoid leukaemia, malignant lymphoma, multiple myeloma, Waldenström's disease, treatment with a cortisone derivative, immunosuppressive treatment, thymus ablation, chemotherapy, radiotherapy, a viral infection, a bacterial infection, an infection caused by a fungus and/or yeast, an infection caused by a parasite, a dietary deficiency, an auto-immune disease, chronic kidney disease, hematologic malignancy, toxic blood disease, asplenia, hyposplenia, a metabolic disease, a cancer affecting the immune system, a drug therapy, a splenectomy, a substance addiction, alcoholism, immunosenescence and combinations thereof, preferably immunosenescence.

Metabolic disease can be selected from among type-2 diabetes, cirrhosis, non-alcoholic hepatic steatosis, obesity, and combinations thereof.

Treatment can be immunosuppressive treatment, a corticoid, chemotherapy, radiotherapy, and combinations thereof.

Advantageously, radiotherapy can be selected from among X- or Gamma-irradiation.

Chemotherapy can be selected from among an antimetabolite, an alkylating agent, a topoisomerase inhibitor, an anthracycline, and combinations thereof.

The alkylating agent can be selected from among dacarbazine, temozolomide, streptozocin, cyclophosphamide, ifosfamide, melphalan, procarbazine, busulfan, triphosphoramide, hexamethylmelamine, chlormethine, platinum salts such as cisplatin, carboplatin, oxaliplatin and combinations thereof. The antimetabolite can be selected from among azathioprine, methotrexate, mycophenolic acid, mycophenolate mofetil, fludarabine, and combinations thereof. The anthracycline can be selected from among doxorubicin, pegylated doxorubicin, daunorubicin, epirubicin, mitoxantrone, pirarubicin, idarubicin, actinomycin D, amsacrine and combinations thereof. The topoisomerase inhibitor can be selected from among topoisomerase 1 inhibitors and topoisomerase 2 inhibitors and combinations thereof. As examples of topoisomerase 1 inhibitor, mention can be made of irinotecan and topotecan. As examples of topoisomerase 2 inhibitors, mention can be made of anthracyclines and etoposide.

In at least one embodiment, the at least one therapeutic agent is an immunosuppressive treatment.

Advantageously, the immunosuppressive treatment can be selected from among an antimetabolite, TNF-alpha inhibitor, interleukin-1 (IL1) inhibitor, a derivative of cortisone, calcineurin inhibitor, rapamycin, anti-CD25 antibody, lymphoablative treatment, and combinations thereof.

Advantageously, the antimetabolite can be selected from among azathioprine, methotrexate, mycophenolic acid, mycophenolate mofetil, fludarabine, and combinations thereof.

Advantageously, the derivative of cortisone is selected from among betamethasone, ciprofloxacin, cortivazol, dexamethasone, fludrocortisone, methylprednisolone, prednisolone, triamcinolone, and combinations thereof.

Advantageously the calcineurin inhibitor can be selected from among cyclosporine, tacrolimus, and combinations thereof.

Advantageously, the lymphoablative treatment can be selected from among chemotherapy, radiotherapy, an alkylating agent, an intercalator, an anthracycline, anti-thymocyte globulins, CD52 monoclonal antibody, OKT3 monoclonal antibody, and combinations thereof.

Advantageously, the immunodeficiency can be characterised by at least one of the markers selected from among neutropenia, lymphopenia, CD4/CD8 ratio lower than 1, and combinations thereof. The CD4+/CD8+ ratio is the ratio between effector T cells (with the surface marker CD4) and cytotoxic T cells (with the surface marker CD8). The CD4+/CD8+ ratio in peripheral blood of adults and mice in good health is about 2:1, and an altered ratio can indicate diseases related to immunodeficiency or autoimmunity. An inverted CD4+/CD8+ ratio (i.e. lower than 1:1) indicates an impaired immune system.

Preferably, the invention is particularly useful for the treatment or prevention of immunosenescence.

A decrease in immunosenescence can be measured by the reduction of a marker selected from among thymic involution, at least one cytokine selected from among IL1, IL2, IL6, IL12, I15, IL18, IL22; TNF-alpha, Interferon gamma, an increase of at least one cytokine selected from among IL1-Ralpha, IL4, IL10, TGF-beta 1, reactive C-protein, the number of resident senescent T cells in the spleen, the level of circulating IgG immunoglobulin produced by memory B cells, the level of circulating IgA immunoglobulin produced by memory B cells, and combinations thereof. Thymic involution corresponds to a reduced volume of the thymus. A decrease in immunosenescence can be observed when the size of the thymus increases.

A decrease in immunosenescence can also be measured via the increase of a marker selected from among the production of new naive T cells, the capacity to respond to new antigens, the accumulation of memory T cells, the number of circulating B cells, the level of circulating IgD immunoglobulins produced by naive cells, the level of circulating IgM immunoglobulins produced by naive cells, vaccinal immunogenicity, the CD4/CD8 ratio, IL1-Ra, IL4 level, IL10 level, TGFbeta-1 level, sedimentation rate, and combinations thereof.

In particular, the inventors have shown that the use of the compounds of the invention, preferably compounds I-A and I-B, particularly allow an increase in the size of the thymus, an increase in the number of CD8+ thymocytes, an increase in the number of B lymphocytes in bone marrow, an increase in the number of CD38+ B lymphocytes in bone marrow, an increase in the number of CD45+B lymphocytes in bone marrow, an increase in the number of precursors of B lymphocytes in bone marrow, an increase in the number of CD45+ T cells in the spleen, an increase in the number of B cells activated in the spleen, an increase in the number of memory B cells in the spleen, an increase in the number of germinal B cells in the spleen, an increase in the number of CD4+ T cells in the spleen, an increase in the number CD4+ memory T cells in the spleen, an increase in the number of CD4+ effector T cells, an increase in the number of CD4+ T cells in the spleen, an increase in the number of CD8+ memory T cells in the spleen, an increase in the number of CD8+ effector memory T cells, an increase in the number of CD4+ et CD8+ cells after activation and reduced expression of IL10.

The NMN, a pharmaceutically acceptable precursor thereof, a pharmaceutically acceptable derivative thereof, or a pharmaceutically acceptable salt thereof, can be administered to a human of at least 40 years of age, preferably at least 45 years of age, preferably at least 50 years of age, preferably at least 55 years of age, preferably at least 60 years of age, preferably at least 65 years of age, more preferably at least 70 years of age, further preferably at least 75 years of age, still further preferably at least 80 years of age to treat immunosenescence.

The NMN, a pharmaceutically acceptable precursor thereof, a pharmaceutically acceptable derivative thereof, or a pharmaceutically acceptable salt thereof and the compositions comprising the same can be administered to a human of at least 15 years of age, preferably at least 20 years of age, more preferably at least 25 years of age, further preferably at least 30 years of age, still further preferably at least 35 years of age for the prevention of immunosenescence.

These analyses can be performed using any method well known to those skilled in the art, from the serum of whole blood taken from the subject, preferably whole blood.

### Mode of Administering and Pharmaceutical Dosage Form

The nicotinamide mononucleotide (NMN), a pharmaceutically acceptable precursor thereof, a pharmaceutically acceptable derivative thereof, or a pharmaceutically acceptable salt thereof, and the compositions comprising the same can be administered via oral, ocular, sublingual, parenteral, transcutaneous, vaginal, peridural, intravesical, rectal or inhalation route.

Preferably, the nicotinamide mononucleotide (NMN), a pharmaceutically acceptable precursor thereof, a pharmaceutically acceptable derivative thereof, or a pharmaceutically acceptable salt thereof, can be administered via oral or parenteral route, preferably via oral route.

Preferably, the parenteral route can be selected from among the intra-arterial route, intravenous route, intramuscular route, subcutaneous route, intraperitoneal route, the intravenous route being particularly preferred.

Preferably, the NMN and the composition of the invention are administered via oral route.

The composition of the invention can be in the form of a tablet, hard capsule, sachet, granule, soft shell capsule, lyophilizate, lozenge, suspension, gel, syrup, solution, water/oil emulsion, oil/water emulsion, oil, cream, milk, spray, ointment, phial, suppository, eye drops, vaginal ovula, vaginal capsule, liquid for inhalation, dry powder inhalator, pressurized inhalator with metering valve. Preferably, the composition of the invention is in the form of a gastro-resistant soft shell capsule or sublingual tablet.

By «gastro-resistant» it is meant a dosage form which does not dissolve in the stomach. Said dosage forms have delayed release i.e. they have a coating or coating composition that resists the acid pH of the stomach (pH<2) to dissolve in the intestine. A gastro-resistant property is determined following the test laid down by the European Pharmacopoeia. In brief, the gastro-resistant property of a capsule is measured in 0.1 M hydrochloric acid at 37° C. as disintegration medium in disintegrating apparatus. This medium imitates the physicochemical conditions of the stomach. The capsules are incubated in this medium for 1 h. The capsule must not exhibit any signs of disintegration or cracking which could lead to loss of content. The capsule is then incubated for 1 h in phosphate buffer solution of pH 6.8 at 37° C., this solution imitating the conditions of the intestinal medium in accordance with the recommendations of the European Pharmacopeia. The capsule must be fully disintegrated in less than one hour.

By «sublingual tablet» it is meant a dosage form to be placed under the tongue so that the active ingredient is absorbed by sublingual mucosa, and in particular by the ranine vein and artery.

The dosage form of the composition of the invention can also have immediate release: said dosage form allows rapid absorption of the NAD precursor and therefore shorter time of action. Immediate-release dosage forms are particularly dispersible, orodispersible, effervescent tablets and oral lyophilizates.

The dosage form of the composition of the invention can also have delayed release. The dissolution and absorption of the NAD precursor take place in the intestines, which limits gastric irritation or degradation of active ingredients sensitive to acid pH. These are mostly gastro-resistant forms i.e. the tablets or granules are coated with a polymeric film that is insoluble in acid medium but permeable to water in an alkaline medium, or of lipid type degraded by intestinal lipases.

The dosage form of the composition of the invention can also have extended and sequential release. Forms having sequential release (released at precise time intervals) and extended release (continuous release of the active ingredient until exhaustion) promote spreading of release of the active ingredient over time to maintain an efficient plasma concentration for a longer length of time in the patient's body.

A suitable dosage level can be about 0.01 to 250 mg/kg per day, about 0.05 to 100 mg/kg per day, or about 0.1 to 50 mg/kg per day. Within this range, the dose can be from 0.05 to 0.5, 0.5 to 5 or 5 to 50 mg/kg per day. For oral administering, the compositions are preferably provided in the form of tablets containing from 1.0 to 1000 milligrams of active ingredient, in particular 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrams of active ingredient for symptomatic adjustment of the dose to the patient being treated. For example, the dosage can be between 100 mg/day and 5000 mg/day, preferably between 500 mg/day and 1000 mg/day. The compounds can be administered with a schedule of 1 to 4 times per day, preferably, once, twice or three times per day. Three times per day is perfectly suitable. The treatment period is dependent on the patient and is determined by the physician. It can range from one day to one year or even longer, preferably from one week to three months, more preferably from two weeks to six weeks. It is to be understood however that the specific dose level and dose frequency as well as the treatment period for a given patient can vary and will depend on various factors in particular the action of the specific compound used, the metabolic stability and action time of this compound, age, body weight, general state of health, gender, diet, mode and time of administering, excretion rate, combination of medication, and the receiver of this treatment.

The NMN, a pharmaceutically acceptable precursor thereof, a pharmaceutically acceptable derivative thereof, or a pharmaceutically acceptable salt thereof can be administered at a daily dose of 10 mg/kg, with a maximum of 1 g/day.

Preferably, the NMN, a pharmaceutically acceptable precursor thereof, a pharmaceutically acceptable derivative thereof, or a pharmaceutically acceptable salt thereof, can be administered at a daily dose of 10 mg/kg, with a maximum of 1 g/day over a period of 10 to 60 days, preferably over a period of 14 to 42 days, more preferably over a period of about 30 days preceding the administering of at least one additional therapeutic agent, preferably a vaccine or vaccine booster.

The NMN, a pharmaceutically acceptable precursor thereof, a pharmaceutically acceptable derivative thereof, or a pharmaceutically acceptable salt thereof, can be administered at Day 1, Day 2, Day 3, Day 4, Day 5, Day 6, Day 7, Day 8, Day 9, Day 10, Day 11, Day 12, Day 13, Day 14, Day 15, Day 16, Day 17, Day 18, Day 19, Day 20, Day 21, Day 22, Day 23, Day 24, Day 25, Day 26, Day 27, Day 28, Day 29, Day 30, Day 31.

The NMN, a pharmaceutically acceptable precursor thereof, a pharmaceutically acceptable derivative thereof, or a pharmaceutically acceptable salt thereof, can be used in an amount of between 5 mg/day and 1000 mg/day.

### Therapeutic Combinations

Advantageously, the NMN and the composition of the invention are used in combination with at least one additional therapeutic agent. In one embodiment, the composition of the invention comprises the NMN or a pharmaceutically acceptable salt thereof, a pharmaceutically acceptable excipient and at least one additional therapeutic agent.

Within the context of the present invention, an «excipient» designates any substance other than the NMN in the composition and not having any therapeutic effect. The excipient does not interact at chemical level with the NMN or with any other additional therapeutic agent.

The NMN, a pharmaceutically acceptable precursor thereof, a pharmaceutically acceptable derivative thereof, or a pharmaceutically acceptable salt thereof, and the compositions comprising the same can be administered in combination with at least one other therapeutic agent.

In at least one embodiment, the at least one other therapeutic agent is a vaccine that can be selected from among attenuated live vaccines and inactivated vaccines.

In one embodiment, said vaccine is selected from among a vaccine against a virus, bacterium, parasite, or yeast and/or fungus, or combinations thereof.

Preferably, said vaccine is a vaccine against a virus selected from among Influenzavirus, Coronavirus, Respirovirus, Pneumovirus, Metapneumovirus, Adenovirus, Enterovirus, Rhinovirus, Hepatovirus, Erbovirus, Aphtovirus, Norovirus, Alphavirus, Rubivirus, Flavivirus, Hepacivirus, Pestivirus, Ebola, Morbillivirus, Rubulavirus, Henipavirus, Arenavirus, Orthobunyavirus, Phlebovirus, Rotavirus, Simplexvirus, Varicellovirus, Papillomavirus, Cytomegalovirus or combinations thereof.

In one embodiment, said vaccine is a vaccine against a bacterium selected from among Pneumococcus, Streptococcus, Corynebacterium, Clostridium, Mycobacterium, Bordetella, Neisseria and combinations thereof.

In one embodiment, said vaccine is a vaccine against a parasite selected from among Schistosoma, Leishmania, Babesia and combinations thereof.

In one embodiment, said vaccine is a vaccine against a yeast and/or fungus selected from among Trichophyton, Toxoplasma, Eimeria, Candida and combinations thereof.

In one embodiment, said vaccine is a vaccine against a bacterium selected from among Pneumococcus, Streptococcus, Corynebacterium, Clostridium, Mycobacterium, Bordetella, Neisseria and combinations thereof.

Preferably, the Mycobacterium is Mycobacterium tuberculosis. Preferably, said Neisseria is Neisseria meningitis.

In one embodiment, said vaccine is a vaccine against a parasite selected from among Schistosoma, Leishmania, Babesia and combinations thereof.

In one embodiment, said vaccine is a vaccine against a yeast and/or fungus selected from among Trichophyton, Toxoplasma, Eimeria, Candida and combinations thereof.

In one embodiment, the at least one therapeutic agent is a radiotherapy, chemotherapy, or combinations thereof.

Advantageously, the radiotherapy can be selected from among X- or Gamma irradiation.

Advantageously, the chemotherapy can be selected from among an antimetabolite, an alkylating agent, a topoisomerase inhibitor, an anthracycline, and combinations thereof such as defined above.

The alkylating agent can be selected from among dacarbazine, temozolomide, streptozocin, cyclophosphamide, ifosfamide, melphalan, procarbazine, busulfan, triphosphoramide, hexamethylmelamine, chlormethine, platinum salts such as cisplatin, carboplatin, oxaliplatin and combinations thereof. The antimetabolite can be selected from among azathioprine, methotrexate, mycophenolic acid, mycophenolate mofetil, fludarabine, and combinations thereof. The anthracycline can be selected from among doxorubicin, pegylated doxorubicin, daunorubicin, epirubicin, mitoxantrone, pirarubicin, idarubicin, actinomycin D, amsacrine and combinations thereof. The topoisomerase inhibitor can be selected from among topoisomerase 1 inhibitors and topoisomerase 2 inhibitors and combinations thereof. As examples of topoisomerase 1 inhibitor, mention can be made of irinotecan and topotecan. As examples of topoisomerase 2 inhibitors, mention can be made of anthracyclines and etoposide.

In at least one embodiment, the at least one therapeutic agent is immunosuppressive treatment.

Advantageously, the immunosuppressive treatment can be selected from among an antimetabolite, TNF alpha inhibitor, interleukin-1 (IL1) inhibitor, a derivative of cortisone, a calcineurin inhibitor, rapamycin, an anti-CD25 antibody, lymphoablative treatment and combinations thereof.

Advantageously, the antimetabolite can be selected from among azathioprine, methotrexate, mycophenolic acid, mycophenolate mofetil, fludarabine, and combinations thereof.

Advantageously, the derivative of cortisone is selected from among betamethasone, ciprofloxacin, cortivazol, dexamethasone, fludrocortisone, methylprednisolone, prednisolone, triamcinolone, and combinations thereof.

Advantageously, the calcineurin inhibitor can be selected from among cyclosporine, tacrolimus, and combinations thereof.

Advantageously, the lymphoablative treatment can be selected from among a chemotherapy, radiotherapy, an alkylating agent, an intercalator, an anthracycline, anti-thymocyte globulins, CD52 monoclonal antibody, OKT3 monoclonal antibody and combinations thereof.

The excipient can be selected from among a filler agent, a lubricant, flavouring, colouring agent, an emulsifier, compression agent, diluent, preserving agent, gelling agent, plasticizer, surfactant, or combinations thereof. Those skilled in the art are able to select the excipient as a function of the chosen dosage form.

The composition of the invention can be a pharmaceutical composition. In this case, the excipient is a pharmaceutically acceptable excipient such as defined above.

The composition of the invention can also be a food supplement.

### NMN Derivatives and Precursors

In the invention, the NMN derivative can be selected from among alpha nicotinamide mononucleotide (α-NMN), dihydronicotinamide mononucleotide (denoted NMN-H), the compound of formula (I):

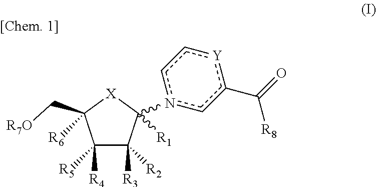

or one of the pharmaceutically acceptable salts, hydrates, solvents, or crystals thereof, where:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are each independently selected from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, C₁-C₈ alkyl, C₁-C₈ thio-alkyl, C₁-C₈ heteroalkyl and OR; wherein R is selected from among H and C₁-C₈ alkyl;
R₇ is selected from among H, P(O)R9R10, P(S)R9R10 and

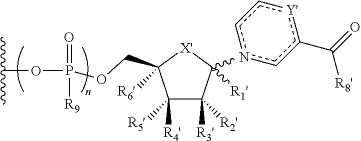

wherein n is an integer equal to 1 or 3; wherein
R₉ and R₁₀ are each independently selected from among OH, OR₁₁, NHR₁₃, NR₁₃R₁₄, a (C₁-C₈) alkyl, a (C₂-C₈) alkenyl, a (C₂-C₅)alkynyl, a (C₃-C₁₀) cycloalkyl, a (C₅-C₁₂) aryl, (C₁-C₈)alkyl aryl, (C₁-C₅) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from among 1 to 8; and P(O)(OH)OP(O)(OH)₂;
R₁₂ is selected from among H, C₁-C₁₀ alkyl, C₁-C₈ alkenyl, C₁-C₈ alkynyl, C₁-C₁₀ haloalkyl, C₃-C₁₀ cycloalkyl, C₃-C₁₀ heterocycloalkyl, C₅-C₁₈aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀, together with the phosphorus atoms to which they are attached, form a 6-membered ring in which —R₉-R₁₀— represents —CH—CH—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
   R₉ and R₁₀, together with the phosphorus atoms to which they are attached, form a 6-membered ring in which —R₉-R₁₀— represents —O—CH—CH—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃ and halogen; where R₁₃ and R₁₄ are each independently selected from among H, (C₁-C₈) alkyl and (C₁-C₈) alkyl aryl;
Y is selected from among CH, CH₂, C(CH₃)₂ et CCH₃;
[symbol] represents a single or double bond along Y; and
[symbol] represents the alpha or beta anomer depending on the position of R₁
   or one of the stereoisomers, salts, hydrates, solvents or crystals thereof
      or
      the compound of formula (Ia):

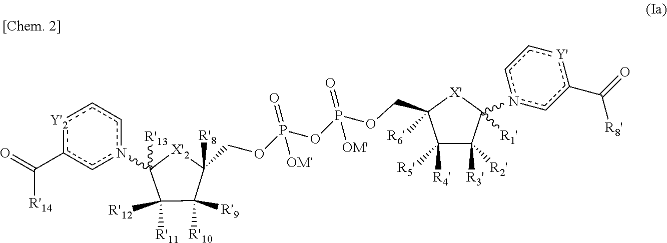

or one of the stereoisomers, salts, hydrates, solvents, or crystals thereof, in which:
X′₁ and X′₂ are independently selected from among 0, CH₂, S, Se, CHF, CF₂, and C═CH₂;
R′₁ and R′13 are independently selected from among H, azido, cyano, a C₁-C₈ alkyl, a C₁-C₈ thio-alkyl, a C₁-C₈ heteroalkyl, and OR, wherein R is selected from H and a C₁-C₈ alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH₂ group; wherein R_{AA} is a side chain selected from a proteogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;
Y′1 and Y′2 are independently selected from among CH, CH₂, C(CH₃)₂ or CCH³;
M′ is selected from among H or a suitable counter ion;
[symbol] represents a single or double bond depending on Y′1 and Y′2; and
[symbol] represents an alpha or beta anomer depending on the position of R′₁ and R′₁₃;
   and combinations thereof.

In a first preferred embodiment, the pharmaceutically acceptable derivative is the compound having the formula (I).

In one variant of the first embodiment, X represents an oxygen.

In one variant of the first embodiment, R₁ and R₆ are each independently a hydrogen.

In one variant of the first embodiment, R₂, R₃, R₄ and R₅ are each independently a hydrogen or an OH.

In one variant of the first embodiment, Y represents a CH.

In one variant of the first embodiment Y represents a CH₂.

In one variant of the first embodiment, R₇ represents a hydrogen.

In one variant of the first embodiment, R₇ represents P(O)(OH)₂.

In one variant of the first embodiment, the compound of the invention is selected from among the compounds having the formula I-A to I-J:

TABLE 1

| Compounds | |
|---|---|
| (anomers) | Structure |
| I-A (beta) | 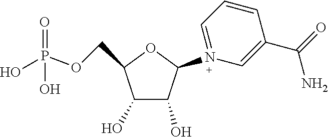 |
| I-B (alpha) | 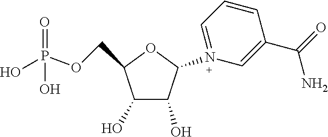 |
| I-C (beta) | 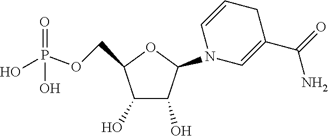 [SMILES: NC(=O)C1=CN([C@@H]2O[C@H](COP(=O)(O)O)[C@@H](O)[C@H]2O)C=CC1] |
| I-D (alpha) | 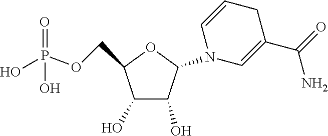 [SMILES: NC(=O)C1=CN([C@H]2O[C@H](COP(=O)(O)O)[C@@H](O)[C@H]2O)C=CC1] |
| I-E (beta) | 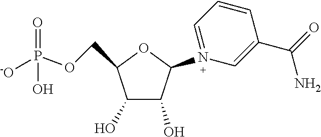 |
| I-F (alpha) | 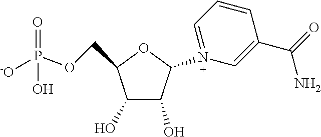 |
| I-G (beta) | 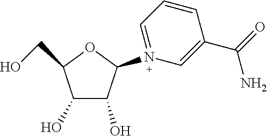 |
| I-H (alpha) | 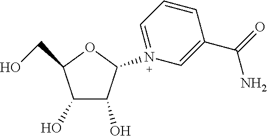 |
| I-I (beta) | 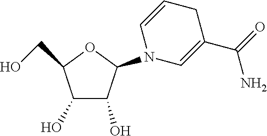 [SMILES: NC(=O)C1=CN([C@@H]2O[C@H](CO)[C@@H](O)[C@H]2O)C=CC1] |
| I-J (alpha) | 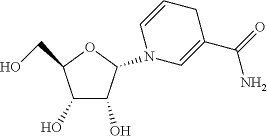 [SMILES: NC(=O)C1=CN([C@H]2O[C@H](CO)[C@@H](O)[C@H]2O)C=CC1] |

In one preferred variant of the first embodiment, the pharmaceutically acceptable derivative is alpha-NMN having the formula:

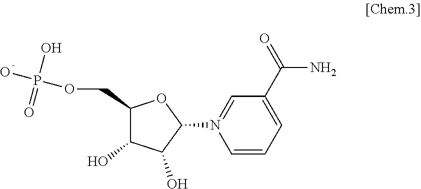

In a preferred second embodiment, the pharmaceutically acceptable derivative is the compound having the formula (Ia).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11 and R′12 each independently represent a hydrogen.

In one variant of the second embodiment, R′2, R′3, R′4, R′5, R′9, R′10, R′11 and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound of the invention is selected from among the compounds having the formula Ia-A to Ia-I:

TABLE 2

| Compounds | |
|---|---|
| (anomers) | Structure |
| Ia-A (beta, beta) | 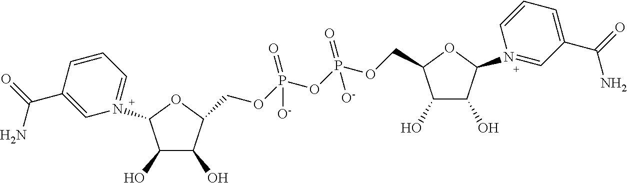 |
| Ia-B (beta, alpha) | 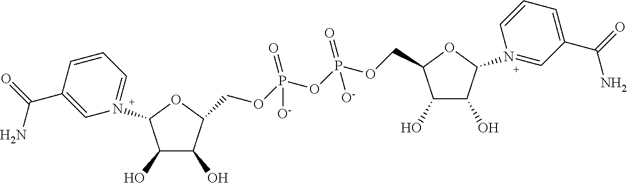 |
| Ia-C (alpha, alpha) | 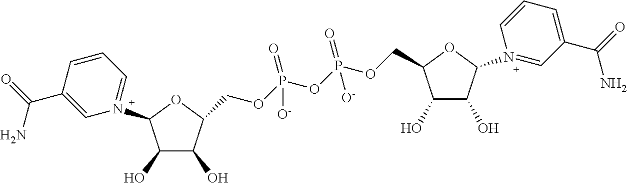 |
| Ia-D (beta, beta) | 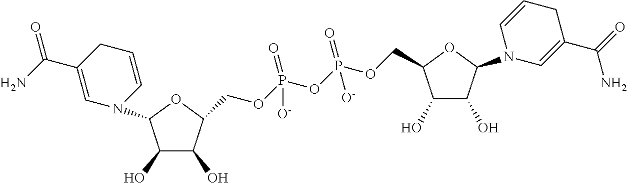 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 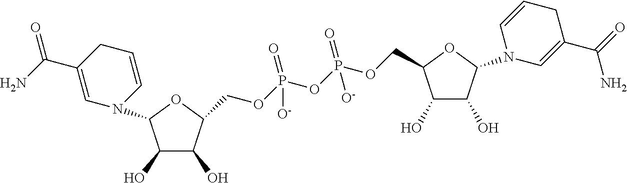 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 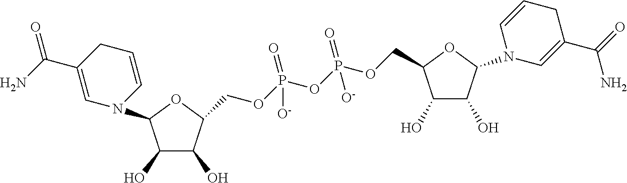 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 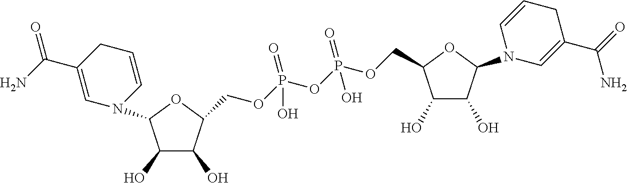 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 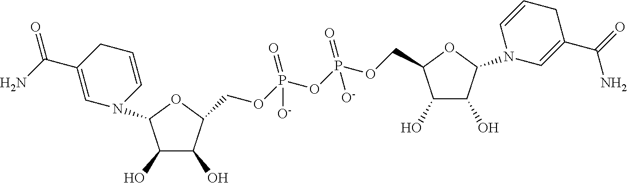 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 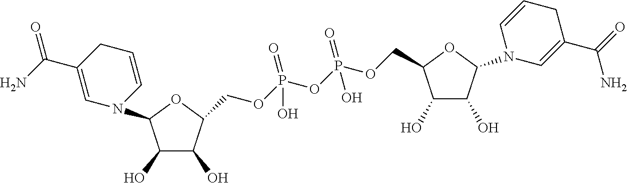 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

In a preferred fourth embodiment, the pharmaceutically acceptable derivative is NMN-H:

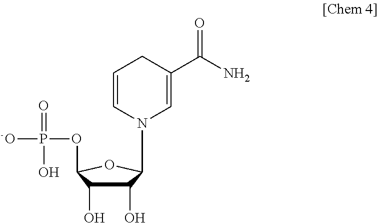

Advantageously, the pharmaceutically acceptable precursor is nicotinamide riboside (denoted NR):

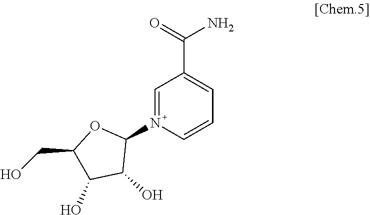

or dihydronicotinamide riboside (denoted —NR—H) of formula:

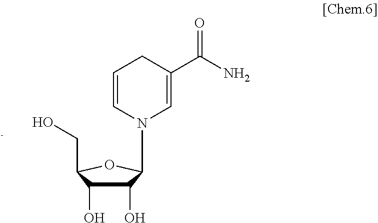

or the combination thereof. Preferably, the precursor is nicotinamide riboside (NR).

Preferably, the derivative of NMN is dihydronicotinamide mononucleotide (NMN-H) and/or alpha-NMN.

### Method for Preparing the Compounds Having the Formula (I) and (La)

The derivatives having the formula (I) or formula (Ia) can be prepared according to any method well known to those skilled in the art.

Method for preparing the compounds of formula (I).

The derivatives of formula can be prepared according to the method described in international patent application WO 2017/024255A1 and patent U.S. Pat. No. 10,611,790 B2.

In particular, the derivatives having the formula (I) as well as alpha-NMN can be prepared according to the method described below.

In particular, the compounds of formula (I) disclosed herein can be prepared as described below from the substrates A-E. It is to be understood by those skilled in the art that these reaction schemes are by no means intended to be limiting and that variations thereto may be made without departing in spirit and scope from the present invention.

According to one embodiment, the invention relates to a method for preparing compounds of formula (I) such as described above.

The method, at a first step, involves the mono-phosphorylation of a compound having the formula (A), in the presence of phosphoryl chloride and a trialkyl phosphate, so as thereby to yield the phosphorodichloridate having the formula (B),

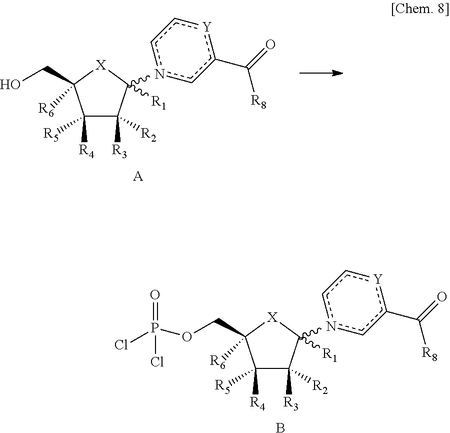

In which X, R₁, R₂, R₃, R₄, R₅, R₆, R₈, Y, [symbol] and [symbol] are such as defined above for the compounds having the formula (I).

At a second step, the phosphorodichloridate having the formula (B) is hydrolysed so as thereby to yield the phosphate having the formula (C),

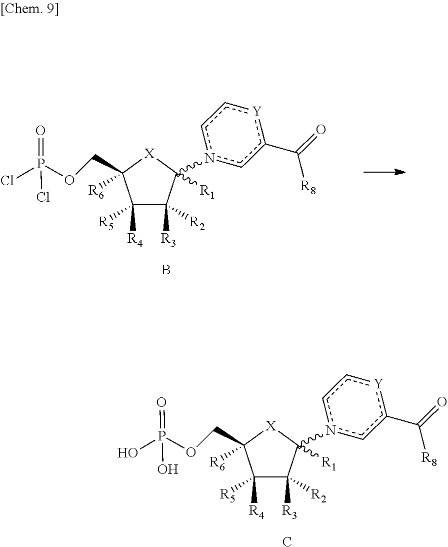

In which X, R₁, R₂, R₃, R₄, R₅, R₆, R₈, Y, [symbol] and [symbol] are as defined hereabove for the compounds having the formula (I).

According to one embodiment, the compound having the formula (A) is synthesised by means of various methods known to those skilled in the art.

According to one embodiment, the compound having the formula (A) is synthesised by reaction of the pentose having the formula (D) with a nitrogenous derivative having the formula (E), in which R, R₂, R₃, R₄, R₅, R₆, R₇, Y, are as described hereabove for the compounds having the formula I, so as thereby to yield the compound having the formula (A-1) which is then selectively deprotected in order to give the compound having the formula (A),

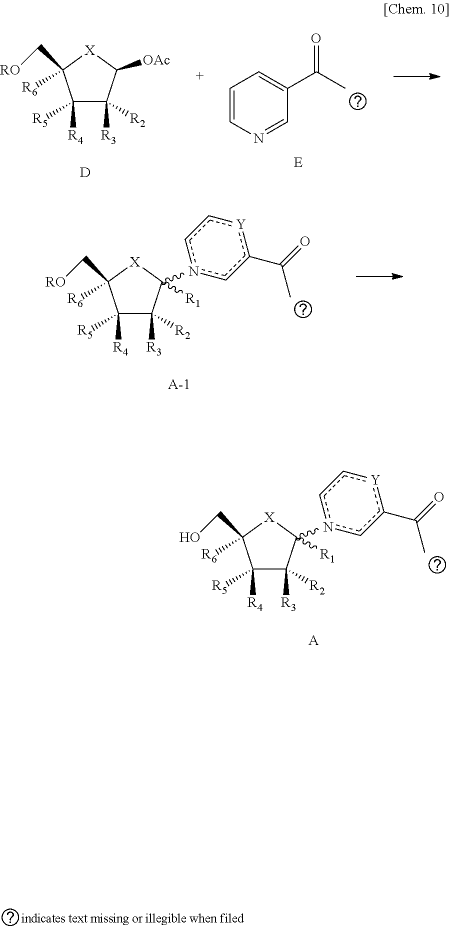

In which X, R₁, R₂, R₃, R₄, R₅, R₆, R₈, Y, [symbol] and [symbol] are such as defined above for the compounds having the formula (I).

According to one embodiment, R is a suitable protecting group known to those skilled in the art. In one embodiment, the protecting group is selected from among triarylmethyls and/or silyls. Without limitation, some examples of triarylmethyl include trityl, monomethoxytrityl, 4,4′-dimethoxytrityl, and 4,4′,4″-trimethoxytrityl groups. Without limitation, some examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl.

According to one embodiment, any hydroxyl group attached to the pentose is protected by an appropriate protecting group known to those skilled in the art.

The selection and exchanging of the protecting groups are well within the scope of knowledge and expertise of those skilled in the art. The protecting groups may also be removed by methods well known to those skilled in the art, for example, with an acid (for example, an inorganic or organic acid), a base or a fluoride source.

In one preferred embodiment, the nitrogenous derivative having the formula (E) is coupled to the pentose having the formula (D) by a reaction in the presence of a Lewis acid so as to thereby yield the compound having the formula (A-1). Without limitation, some examples of Lewis acids include Trimethylsilyl Trifluoromethanesulfonate (TMSOTf), BF₃.OEt₂, TiCl₄ and FeCl₃.

In one embodiment, the method of the present invention additionally also comprises a reduction step of reducing the compound having the formula (A) by various methods well known to those skilled in the art, so as thereby to yield the compound having the formula (A′) in which is CH₂, and R₁, R₂, R₃, R₄, R₅, R₆, R₈, Y, [symbol] and [symbol] are as defined here above for the compounds having the formula (I).

In one particular embodiment, the present invention relates to a method for preparing compounds having the formula I-A, I-C, I-E, I-G.

At a first step, the nicotinamide having the formula E is coupled to the ribose tetraacetate having the formula D by a coupling reaction in the presence of a Lewis acid, so as thereby to yield the compound having the formula A-1:

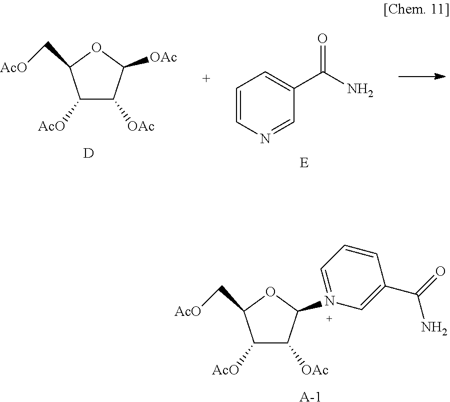

At a second step, an ammoniacal treatment of the compound having the formula A-1 is carried out, so as thereby to yield the compound having the formula I-A:

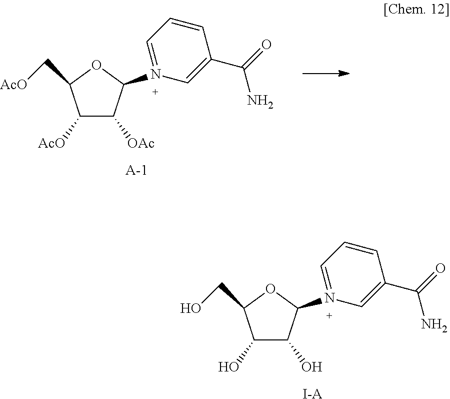

At a third step, the mono-phosphorylation of the compound having the formula I-A, in the presence of phosphoryl chloride and a trialkyl phosphate, thereby yields the phosphorodichloridate having the formula I-A′

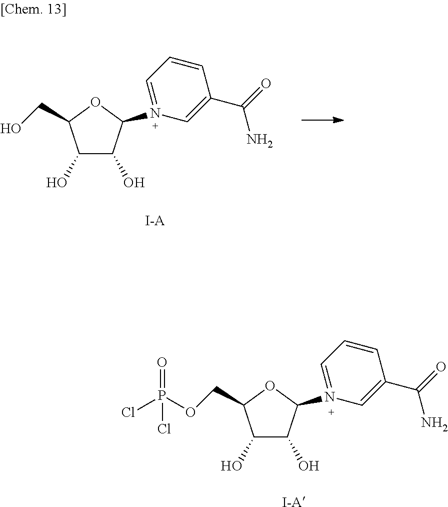

At a fourth step, the phosphorodichloridate having the formula B is hydrolysed so as thereby to yield the compound having the formula I-C:

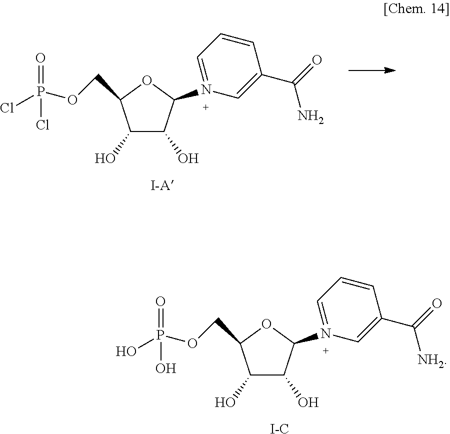

In one embodiment, a reduction step of reducing the compound having the formula I-A is carried out, so as thereby to yield the compound having the formula I-E.

The compound having the formula I-E is then mono-phosphorylated as described in the fourth step and hydrolysed so as thereby to yield the compound having the formula I-G.

According to one embodiment, the compounds having the formula (I) are selected from compounds I-A to I-J in the table below:

TABLE 1

| Compounds | |
|---|---|
| (anomers) | Structure |
| I-A (beta) | 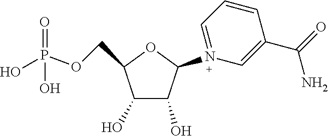 |
| I-B (alpha) | 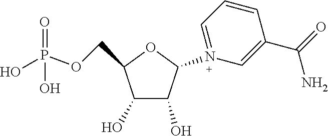 |
| I-C (beta) | 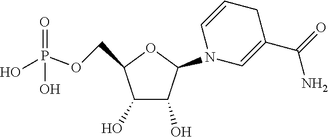 [SMILES: NC(=O)C1=CN([C@@H]2O[C@H](COP(=O)(O)O)[C@@H](O)[C@H]2O)C=CC1] |
| I-D (alpha) | 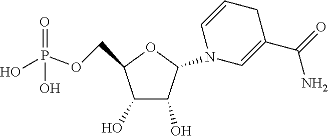 [SMILES: NC(=O)C1=CN([C@H]2O[C@H](COP(=O)(O)O)[C@@H](O)[C@H]2O)C=CC1] |
| I-E (beta) | 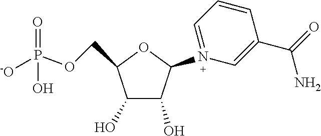 |
| I-F (alpha) | 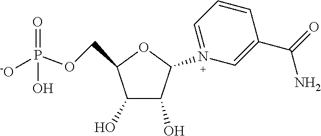 |
| I-G (beta) | 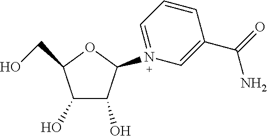 |
| I-H (alpha) | 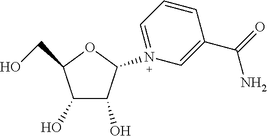 |
| I-I (beta) | 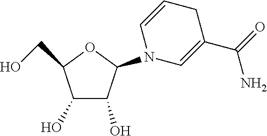 [SMILES: NC(=O)C1=CN([C@@H]2O[C@H](CO)[C@@H](O)[C@H]2O)C=CC1] |
| I-J (alpha) | 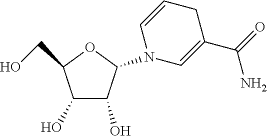 [SMILES: NC(=O)C1=CN([C@H]2O[C@H](CO)[C@@H](O)[C@H]2O)C=CC1] |

In one preferred embodiment, the compounds of the invention are the compounds of formula I-A, I-C, I-E et I-G in the Table above, or a pharmaceutically acceptable salt and/or solvate thereof. In one further preferred embodiment, the compound is the compound of formula I-C or I-D or a pharmaceutically acceptable salt and/or solvate thereof.

Synthesis of the formula 1 compounds in which R7 is

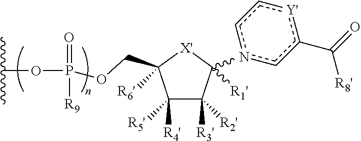

and n=1:

At a first step, the synthesis may comprise the phosphorylation of alpha-nicotinamide riboside, in the presence of phosphorus chloride and a trialkyl phosphate, to obtain a phosphorodichloridate:

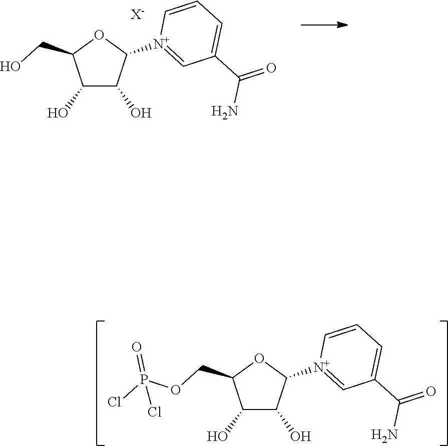

The compound I-B (alpha NMN) is then added:

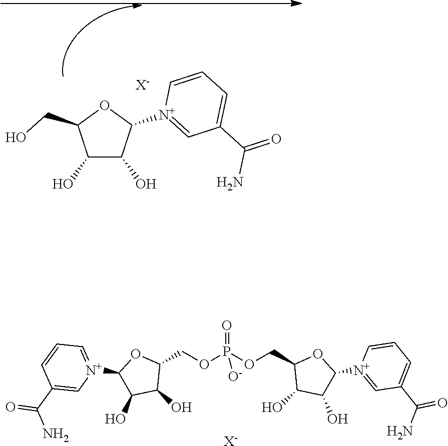

Alternatively, the compound of the invention can be prepared via activation of compound I-B through the addition of carbonyldiimidazole (CDI):

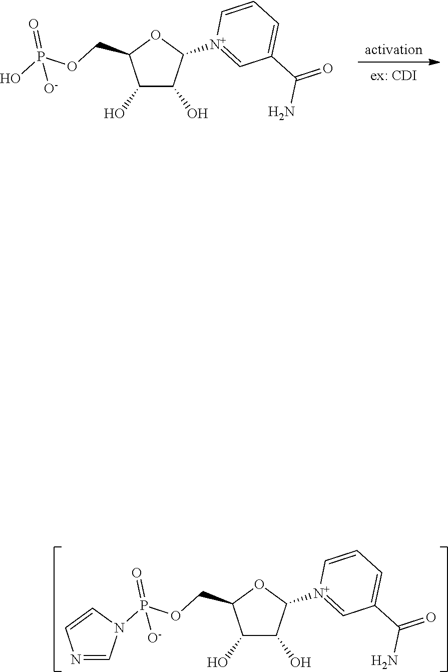

To which alpha-nicotinamide riboside is then added as follows:

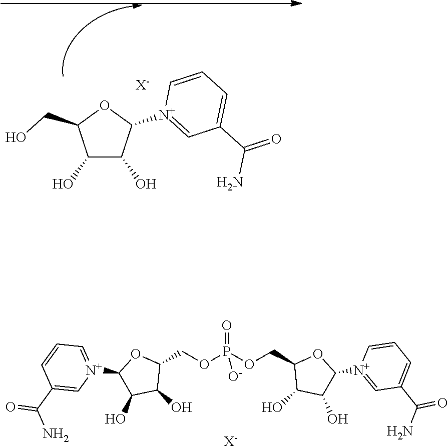

Synthesis of the formula I compounds in which R7 is

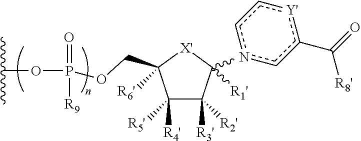

and n=3:
A compound of formula I comprising three phosphate groups can be prepared as follows. Compound I-B can be prepared through the addition of carbonyldiimidazole (CDI):

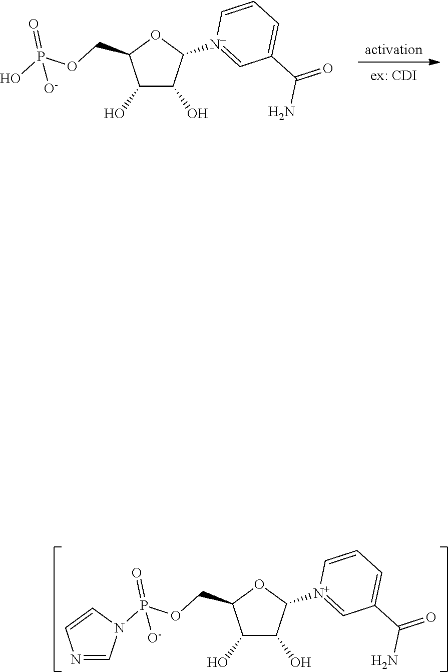

To which tertbutylamine-phosphate is added:

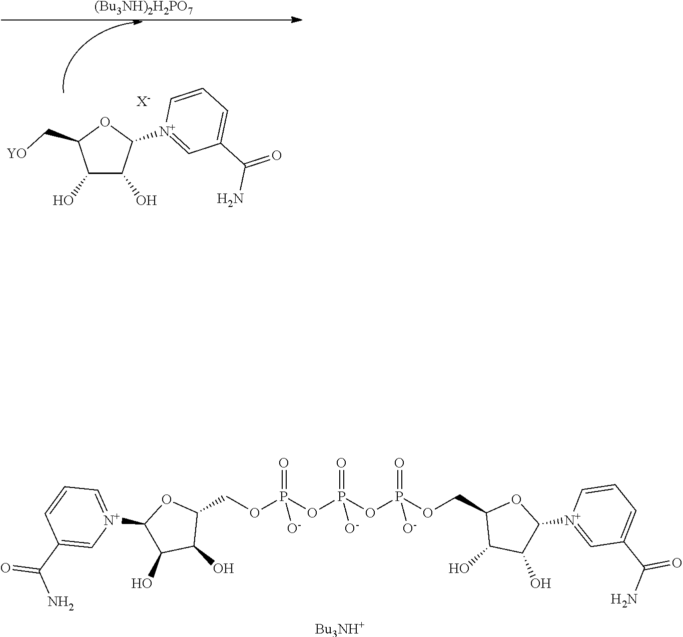

Method for Preparing the Derivatives Having the Formula (Ia)

In particular, the compounds having the formula Ia presented herein may be prepared as described here below from the substrates X-XIII. It is to be understood by the skilled in the art that these diagrams are by no means intended to be limiting and that variations thereto in terms of the detail may be made without departing in spirit and scope from the present invention.

According to one embodiment, the invention relates to a compound preparation method for preparing the compound having the formula I described hereabove.

The method consists first of all in mono-phosphorylating a compound having the formula X, in the presence of phosphoryl chloride in a trialkyl phosphate, in order to obtain the compound phophorodichloridate XI,

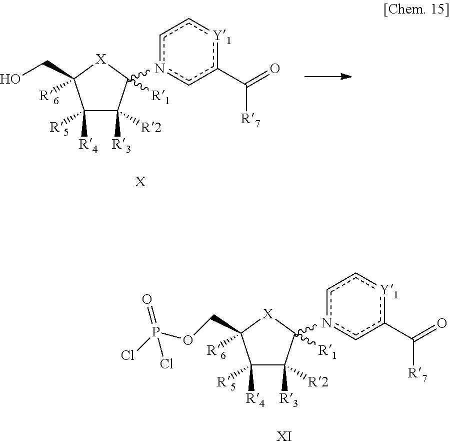

in which X′₁, R′₁, R′₂, R′₃, the R′₄, R′₅, R′₆, R′₇, Y′₁, [symbol] and [symbol] are such as defined above.

At a second step, the hydrolysis of the phosphorodichloridate XI obtained at the first step gives the phosphate compound having the formula XII,

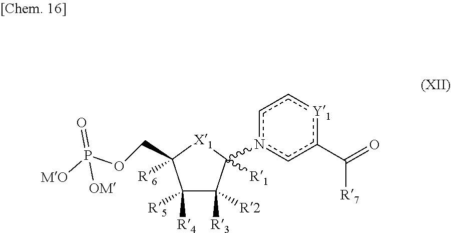

In which X′₁, R′₁, R′₂, R′₃, R′₄, R′₅, R′₆, R′₇, Y′₁, M′, [symbol] and [symbol] are such as defined above.

The phosphate compound having the formula XII obtained at the second step is then reacted with a phosphorodichloridate compound having the formula XIII obtained as described at the first step,

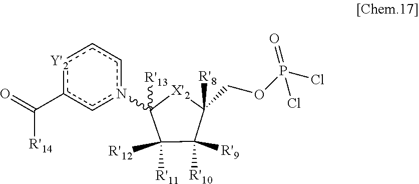

in which X′₂, R′₈, R′₉, R′₁₀, R′₁₁, R′₁₂, R′₁₃, R′₁₄, Y′₂, [symbol] and [symbol] are such as described herein for formula Ia, in order to give the compound having the formula Ia such as described herein.

According to one embodiment, the method further comprises a reduction step of reducing the compound having the formula Ia, using various methods known to specialists, in order to give the compound having the formula Ia, where Y′₁ and Y′₂ are identical and each represent CH₂, and where X′₁, X′₂, R′₁, R′₂, R′₃, R′₄, R′₅, R′₆, R′₇, R′₈, R′₉, R′₁₀, R′₁₁, R′₁₂, R′₁₃, R′₁₄, Y′₁, Y′₂, and [symbol] , [symbol] are as described herein for formula Ia.

In one variant, R is a suitable protecting group known to the skilled in the art. Triarylmethyl and/or silyl groups are examples of suitable protecting groups. Without limitation, some examples of triarylmethyl include trityl, monomethoxytrityl, 4,4′-dimethoxytrityl, and 4,4′,4″-trimethoxytrityl. Without limitation, some examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl.

According to one representation, any hydroxy group attached to the pentose ring is protected by a suitable protecting group known to the skilled in the art.

The selection and exchanging of the protecting groups are well within the scope of knowledge and expertise of the skilled in the art. Any protecting group may also be removed by methods known in the art, for example, with an acid (for example, an inorganic or organic acid), a base or a fluoride source.

According to one preferred embodiment, the nitrogen compounds having the formula XV are added to the pentose XIV by a coupling reaction in the presence of a Lewis acid in order to give the compound having the formula X-1. Without limitation, some examples of suitable Lewis acids include Trimethylsilyl Trifluoromethanesulfonate (TMSOTf), BF₃.OEt₂, TiCl₄ and FeCl3.

According to one specific embodiment, the invention relates to a compound preparation method for preparing the compound having the formula VIII,

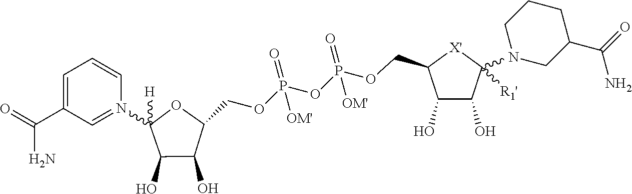

or the pharmaceutically acceptable salts and/or solvates thereof.

At a first step, the nicotinamide having the formula XV is added to the ribose tetraacetate XIV, by a coupling reaction in the presence of a Lewis acid, in order to give the compound having the formula X-1:

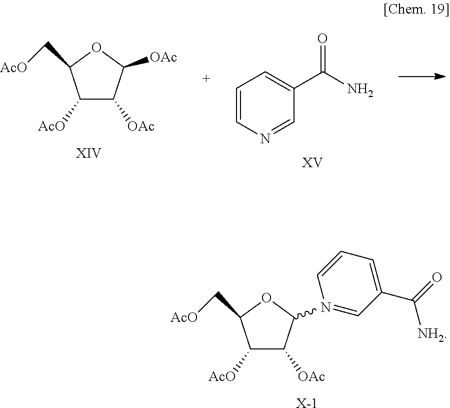

At a second step, an ammoniacal treatment of the compound having the formula X-1 gives the compound having the formula X:

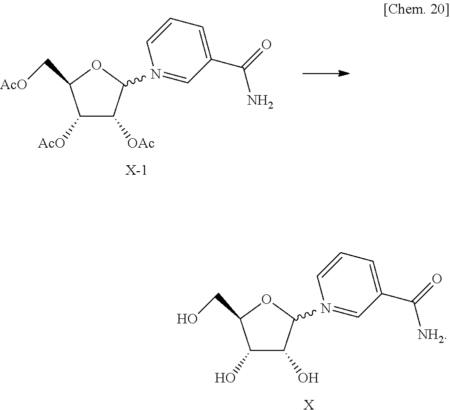

At a third step, the mono-phosphorylation of a compound having the formula X, in the presence of phosphoryl chloride in a trialkyl phosphate, gives the compound phosphorodichloridate XI:

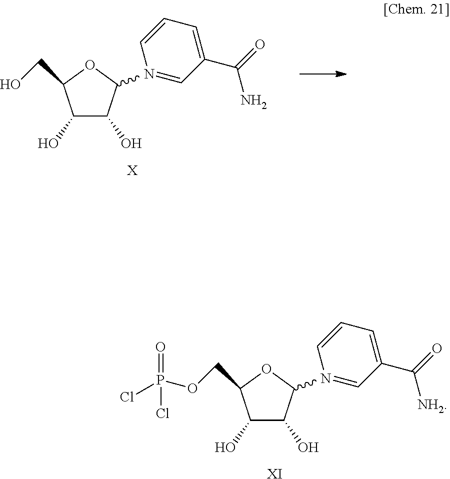

At a fourth step, the phosphorodichloridate compound XI obtained at the third step is partially hydrolysed in order to give the phosphate compound having the formula XII:

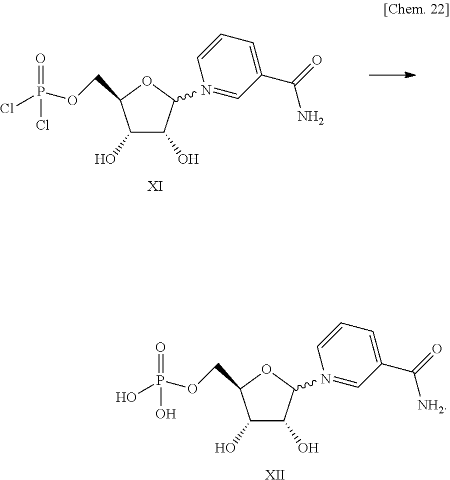

At a fifth step, the phosphate compound having the formula XII obtained at the fourth step is then reacted with the phosphorodichloridate compound having the formula XI obtained as described at the third step, in order to obtain the compound having the formula VIII.

According to another specific implementation embodiment, the invention relates to a compound preparation method for preparing the compound having the formula IX,

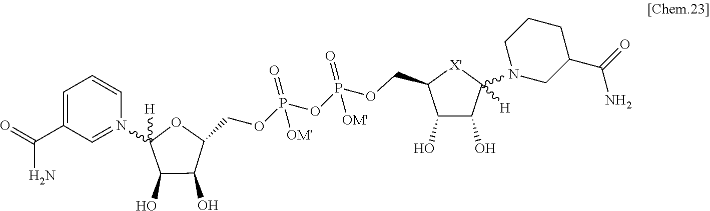

or the pharmaceutically acceptable salts and/or solvates thereof.

According to one variant, the compound having the formula IX is obtained from the compound having the formula VIII, which is synthesised beforehand as described hereabove.

In this embodiment, the compound having the formula IX is obtained by reducing the compound having the formula VIII, using a suitable reducing agent known to the specialised skilled in the art, in order to give the compound having the formula IX.

According to one embodiment, the preferred compounds of the invention are the compounds Ia-A to Ia-I in Table 2:

TABLE 2

| Compounds | |
|---|---|
| (anomers) | Structure |
| Ia-A (beta, beta) | 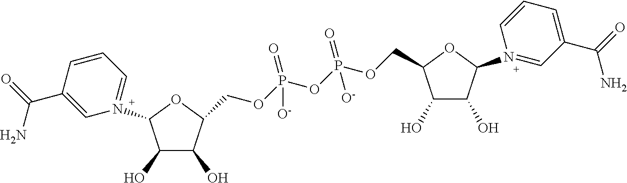 |
| Ia-B (beta, alpha) | 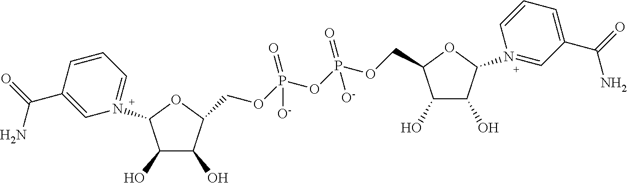 |
| Ia-C (alpha, alpha) | 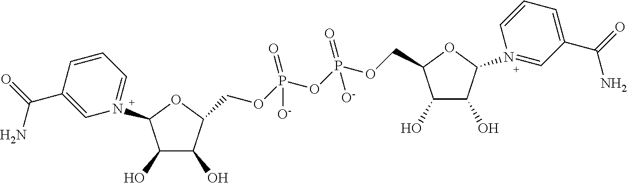 |
| Ia-D (beta, beta) | 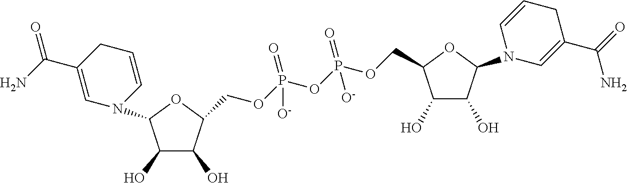 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 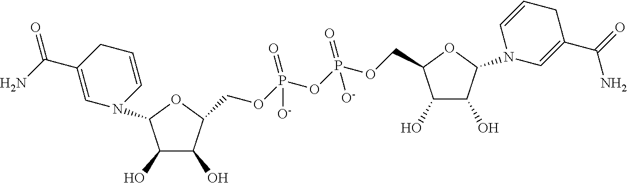 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 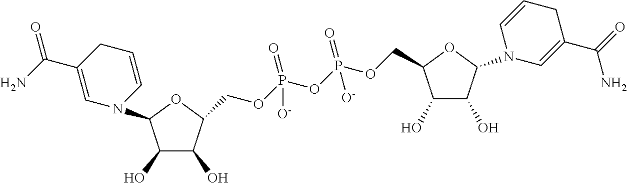 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 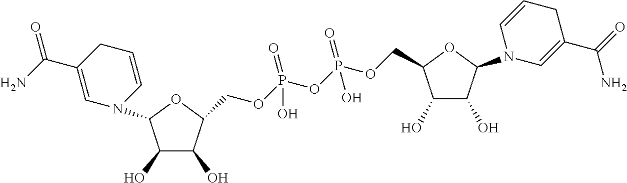 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 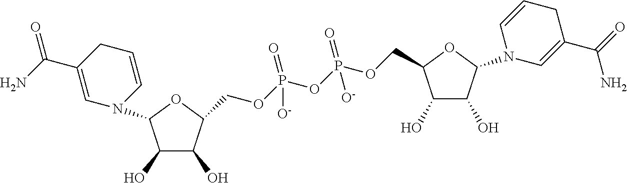 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 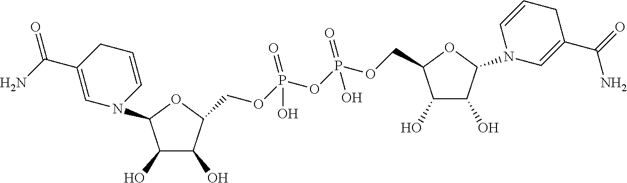 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

### FIGURES

FIG. 1 is a graph showing the change in weight gain (FIG. 1A) and water consumption (FIG. 1B) in groups 1 to 4.

FIG. 2 is a graph showing the change in the weight and size of the thymus in groups 1 to 4.

FIG. 3 is a graph showing the number of thymocytes in groups 1 to 4.

FIG. 4A is a graph showing the number of B cells in the bone marrow samples taken from the mice in groups 1 to 4.

FIG. 4B is a graph showing the number of CD38+ B cells in the bone marrow samples taken from the mice in groups 1 to 4.

FIG. 5A is a graph showing the number of CD45+ B cells in the bone marrow samples taken from the mice in groups 1 to 4.

FIG. 5B is a graph showing the number of pre-pro-B cells in the bone marrow samples taken from the mice in groups 1 to 4.

FIG. 5C is a graph showing the number of pro-B cells in the bone marrow samples taken from the mice in groups 1 to 4.

FIG. 5D is a graph showing the number of pre-B cells in the bone marrow samples taken from the mice in groups 1 to 4.

FIG. 5E is a graph showing the number of immature B cells in the bone marrow samples taken from the mice in groups 1 to 4.

FIG. 6A is a graph showing the number of CD45+ B cells in the spleen samples taken from the mice in groups 1 to 4.

FIG. 6B is a graph showing the number of activated B cells in the spleen samples taken from the mice in groups 1 to 4.

FIG. 6C is a graph showing the number of memory B cells in the spleen samples taken from the mice in groups 1 to 4.

FIG. 6D is a graph showing the number of germinal B cells in the spleen samples taken from the mice in groups 1 to 4.

FIG. 6E is a graph showing the number of plasma B cells in the spleen samples taken from the mice in groups 1 to 4.

FIG. 7A is a graph showing the number of CD4+ T cells in the spleen samples taken from the mice in groups 1 to 4.

FIG. 7B is a graph showing the number of CD4+ memory T cells in the spleen samples taken from the mice in groups 1 to 4.

FIG. 8A is a graph showing the number of naive CD8+ T cells in the spleen samples taken from the mice in groups 1 to 3.

FIG. 8B is a graph showing the number of CD8+ effector T cells in the spleen samples taken from the mice in groups 1 to 3.

FIG. 8C is a graph showing the number of CD8+ effector memory T cells in the spleen samples taken from the mice in groups 1 to 3.

FIG. 8D is a graph showing the number of CD8+ memory T cells in the spleen samples taken from the mice in groups 1 to 3.

FIG. 9A is a graph showing the percentage proliferation of CD4+ T cells compared with the non-stimulated cells in groups 1 to 4.

FIG. 9B is a graph showing the percentage proliferation of CD8+ T cells, compared with the non-stimulated cells in groups 1 to 4.

FIG. 10A is a graph showing the percentage of CD4+ T cells stimulated and not stimulated by beads coated with anti-CD3/CD28 antibodies, producing interferon gamma (IFNγ) in groups 1 to 4.

FIG. 10B is a graph showing the percentage of CD4+ T cells stimulated and not stimulated by beads coated with anti-CD3/CD28 antibodies, producing interleukin 10 (I10) in groups 1 to 4.

### EXAMPLE

In the remainder of the present description, the examples are given to illustrate the present invention and are in no way intended to limit the scope thereof.

### Example 1—Study of the Effects of Compounds I-A (β-NMN) and I-B (α-NMN) on Immunosenescence

The administering of β-NMN at 500 mg/kg, of α-NMN at 500 mg/kg and of the vehicle (water) was obtained via oral route (p.o) in the drinking water to young and old mice. The solutions were prepared by dissolving the powder of β-NMN or α-NMN in the vehicle (water).

The solution was used at ambient temperature for no more than 2 days and was freshly prepared for each new administration. The mice were weighed each week to adapt the dose of the compound to be administered. Throughout the entire phase, a standard food diet and tap water were provided ad libitum.

The study comprised 4 groups each having 6 mice:
Group 1: young mice (aged 10 weeks)+drinking water (vehicle)
Groupe 2: old mice (aged 15 months)+drinking water (vehicle)
Group 3: old mice (aged 15 months)+compound I-B (alpha-NMN) in the drinking water
Group 4: old mice (aged 15 months)+compound I-A (beta-NMN) in the drinking water

The water consumption of the mice was assessed per cage every other day. The mice were weighed every week. The mice in Group 3 were sacrificed after 4 weeks for organ collection and characterisation of T cell function. The mice in Group 4 were sacrificed after 6 weeks for organ collection and characterisation of T cell function. On the day of sacrifice, the mice were anaesthetised with Vetoflurane (isoflurane) and blood samples were taken from the retroorbital sinus. The blood was incubated for 30 minutes at ambient temperature and then centrifuged at 1300 g for 10 minutes.

After sacrificing of the mice, the spleen, thymus, and bone marrow of each animal were removed. The thymus was weighed and measured to evaluate involution thereof. For illustration purposes, photos of the thymus were taken on 5 animals per group. The thymus was divided into two parts: one part was treated with formol for histological examination. The second part was treated for characterisation of the immune cells.

For characterisation of the cells, the thymus and spleen were crushed through a sieve of 40 cells/μm over a 50 mL tube with a syringe plunger. The bone marrow was rinsed with RPMI culture medium using a needle and syringe. The cell suspension was then centrifuged at 400 g for 5 minutes at 4° C. Lysis of the red blood cells was also performed before the cell count and use thereof for cytometry.

The isolated cells of the spleen, thymus and bone marrow were marked with antibodies in accordance with the following tables to identify the proportion of effector and memory naive T and B cells.

The following T cells of the spleen were also examined for characterisation as a function of the combination of proteins on the surface thereof:

TABLE 3

| T cells | Combination of surface proteins |
|---|---|
| Naive T cells | CD25− CD44lo CD62Lhi CD127+ |
| Effector T cells | CD25+ CD44hi CD62Llo CD127− |
| Effector memory T cells | CD25− CD44hi CD62Llo CD127+ |
| Central memory T cells | CD25− CD44hi CD62Lhi CD127+ |
| Senescent CD8+ T cells | CD44hi KLRG1+ CD8+ T |
| Senescent CD4+ T cells | CD4+ PD1+ CD153+ |

The following B cells of the spleen were also examined for characterisation as a function of the combination of proteins on the surface thereof:

TABLE 4

| B cells | Combination of surface proteins |
|---|---|
| Marginal cells in zone B | B220+ CD21hi CD23− CD43− |
| Follicular B cells | CD19+ B220+ CD23+ CD21− CD43− |
| Activated B cells | CD19+ B220+ IgM+ IgD |
| Memory B cells | CD19+ B220+ IgM+ IgG+ IgD− |

The following thymocytes were also examined for characterisation as a function of the combination of proteins on the surface thereof:

TABLE 5

| Thymocytes | Combination of surface proteins |
|---|---|
| Multipotent thymocytes | CD44lo CD25lo CD4− CD8− |
| Double positive thymocytes | CD44lo CD25lo CD4+ CD8+ |
| Single positive thymocytes | CD44lo CD25lo CD4+ CD8− or |
| | CD44lo CD25lo CD4− CD8+ |

The following bone marrow cells were also examined for characterisation as a function of the combination of proteins on the surface thereof:

TABLE 6

| Bone marrow cells | Combination of surface proteins |
|---|---|
| Pre-pro-B cells | CD43+ B220+ IgM− CD19− |
| Pro-B cells | CD43+ B220+ IgM− CD19+ |
| Pre-B cells | CD43− B220+ IgM− CD19+ |
| Immature B cells | CD43− B220+ IgM+ IgD− CD19 |

Proliferation assay: At D0+5 weeks, the splenocytes were marked with 2.5 μM CFSE and cultured at a concentration of 0.25×10⁶ cells per well in a 96-well plate. The cells were stimulated with 2.5 μg InfluvacTetra® and incubated at 37° C. for 96 hours. The cells were then marked with anti-CD4 and CD8 antibodies and the proliferation of the T CD4+ and CD8+ cells was analysed by flow cytometry.

Measured parameters: The results of flow cytometry are expressed in number or as a proportion of cells per organ. The weight of the thymus is expressed in grams.

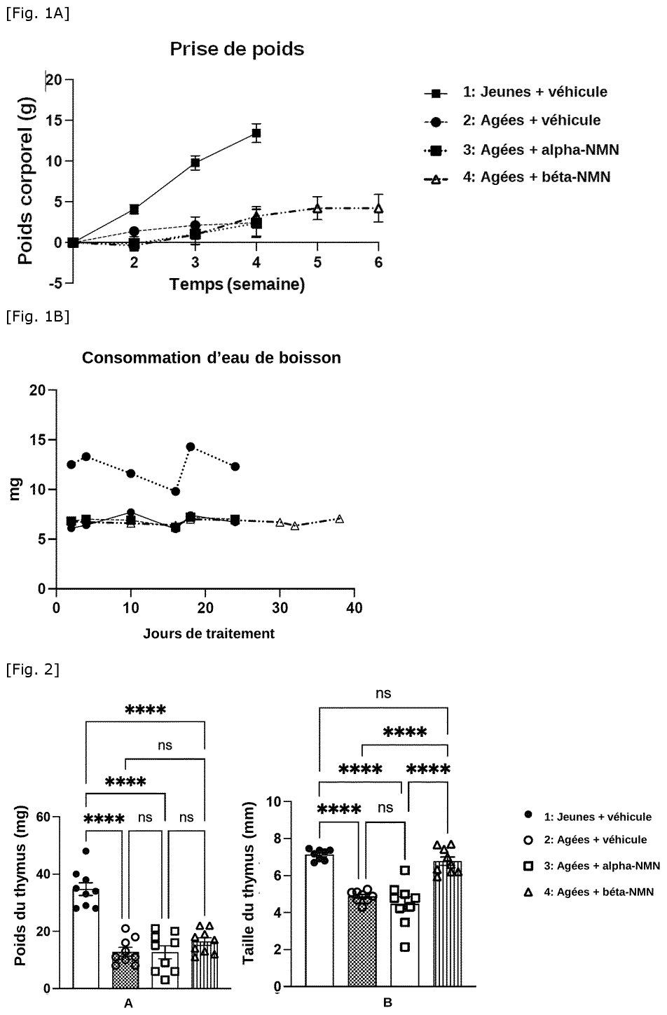

The consumption of compounds I-A and I-B did not significantly increase the weight of the treated mice compared with the mice of 15 months that were not treated (FIG. 1A) and did not have any effect on water consumption compared with the mice of 11 weeks (FIG. 1B).

As shown in FIG. 2, the size of the thymus is considerably and significantly reduced in the old mice (Group 2) compared with the young mice (Group 1), which translates as involution of the thymus. Involution of the thymus with age is seen in humans and validates the model. The administering of alpha-NMN did not allow an increase in the volume or size of the thymus compared with the non-treated old mice. On the other hand, administering of β-NMN did allow a significant increase in the size of the thymus compared with the mice in Groups 2 and 3 up to a level close to that of the young mice in Group 1.

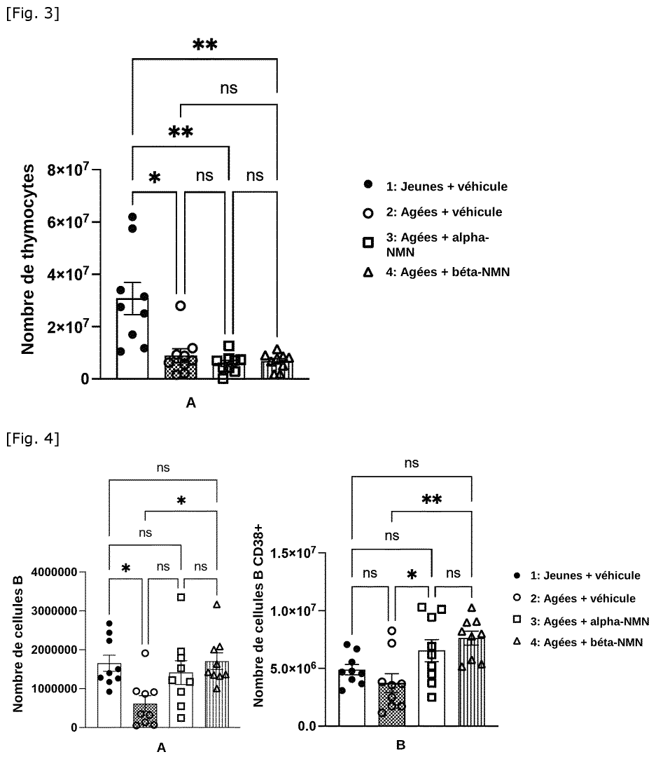

FIG. 3, the total number of thymocytes is reduced in the old mice (Group 2). Solely the treatment with compound 1-A (beta-NMN) allows an increase in the number of CD8+ thymocytes in the old mice.

FIG. 4, the number of B cells is significantly reduced in the old mice (Group 2) compared with the young mice (Group 1) in the bone marrow. The administering of α-NMN (Group 3) and of β-NMN (Group 4) allows a significant increase in the number of B cells and the restoring of an identical or almost identical level to the level observed in the young mice (FIG. 4A). Regarding the CD38+ cells, the level of these cells is decreased in the old mice (Group 2) compared with the young mice (Group 1). The administering of α-NMN (Group 3) and of β-NMN (Group 4) allows the restoring of the number of CD38+ B cells to the level expressed by the young mice in Group 1 (FIG. 4B).

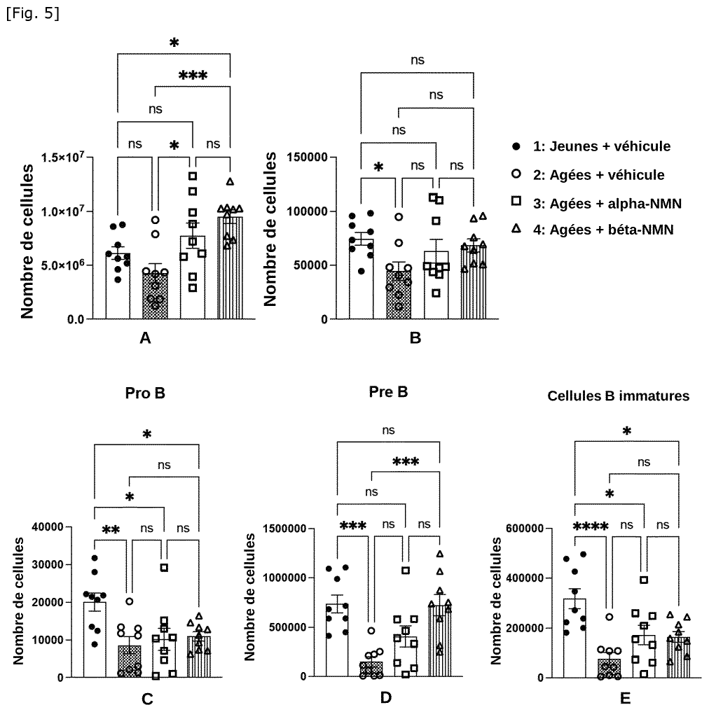

As shown in FIGS. 5A to 5E, the level of B cells is significantly decreased in the old mice in Group 2 compared with the young mice in Group 1. The administering of α-NMN and of β-NMN allows a significant increase in the number of CD45+ B cells up to levels even higher than the level expressed by the mice in Group 1 (see FIG. 5A). The administering of α-NMN and of β-NMN also allows an increase in the number of pre-pro B cells (FIG. 5B) but does not show any impact on the number of pro-B cells (FIG. 5C). On the other hand, the administering of α-NMN (Group 3) and of β-NMN (Group 4) allows an increase in the number of pre-B cells (FIG. 5D) and immature B cells (FIG. 5E).

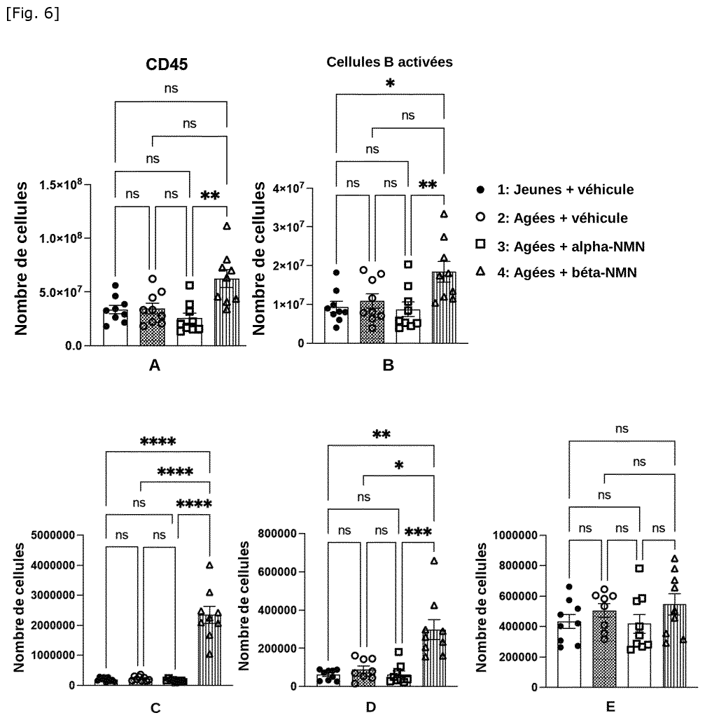

The B cells present in the spleen of the mice in each of the groups was characterized. As shown in FIG. 6A, the number of CD45+ T cells in the spleen is significantly increased through the administering of β-NMN (Group 4) compared with the other groups of mice. The administering of β-NMN also allows an increase in the number of activated B cells in the spleen in significant manner compared with the other groups (FIG. 6B), as well as memory B cells (FIG. 6C) and germinal B cells (FIG. 6D). The number of plasma cells is not modified among the 4 groups (FIG. 6E).

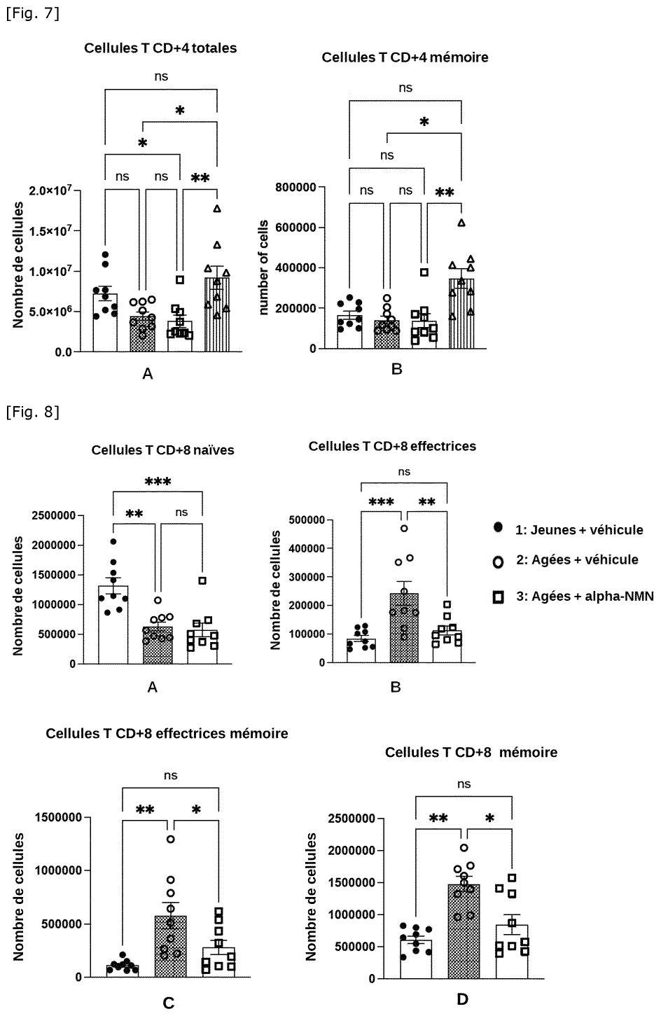

The CD4+ T cells present in the spleen of the mice in each of the groups were characterized. As shown in FIG. 7A, the total number of CD4+ T cells in the spleen is significantly increased through the administering of β-NMN (Group 4) compared with the other groups of mice. The administering of β-NMN also allows an increase in the memory T cells that is significant compared with the other groups (FIG. 7B). The administering d′α-NMN and of β-NMN also allows an increase in the number of CD4+ effector T cells compared with Group 3.

The CD8+ T cells present in the spleen of the mice in Groups 1, 2 and 3 were characterized. As shown in FIG. 8A, the old mice in Group 2 exhibit fewer naive CD8+ T cells than the young mice in Group 1. The administering of α-NMN (Group 3) does not modify the number of naive CD8+ T cells. On the other hand; the administering of α-NMN (Group 3) allows restoring of the levels of effector CD8+ T cells (FIG. 8B), of memory CD8+ T cells (FIG. 8C) and of effector memory CD8+ T cells (FIG. 8D).

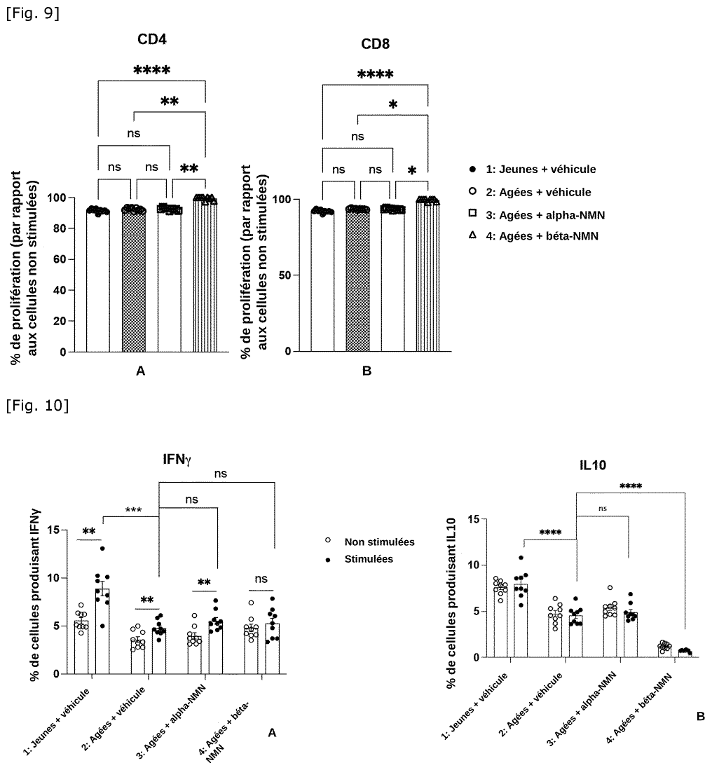

The total splenocytes were stained with Carboxyfluorescein succinimidyl ester (CFSE) and stimulated with beads coated with anti-CD3/CD28 for 96 h. The mean fluorescence intensity of the CFSE was analysed and the ratio before and after stimulation was calculated. As shown in FIGS. 9A and 9B, the proliferation of CD4 and CD8 cells is not impacted by age. However, the treatment with β-NMN allows a significant increase in the proliferation of CD4 and CD8 cells compared with the three other groups of mice.

The total splenocytes were stimulated with beads coated with anti-CD3/CD28 for 18 h. As shown in FIGS. 10A and 10B, ageing reduced the proportion of cells producing IFNγ and IL10 when simulated with beads coated with anti-CD3/CD28. For IFNγ (FIG. 10A), the response to stimulation was lower in the old mice treated with the vehicle or α-NMN, and lack of response was observed in the β-NMN group.

To conclude, the administering of compounds I-A et I-B allows a significant decrease in the signs of immunosenescence.

1. Nicotinamide mononucleotide (NMN), a pharmaceutically acceptable derivative thereof, or a pharmaceutically acceptable salt thereof, for use thereof in the prevention and/or treatment of immunodeficiency, preferably immunosenescence.

2. Nicotinamide mononucleotide (NMN), a pharmaceutically acceptable derivative thereof, or a pharmaceutically acceptable salt thereof, for use thereof according to claim 1, wherein the derivative of NMN can be selected from among alpha nicotinamide mononucleotide (α-NMN), dihydronicotinamide mononucleotide (denoted NMN-H), the compound of formula (I): 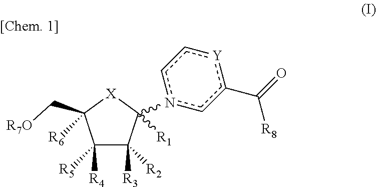
or one the pharmaceutically acceptable stereoisomers, salts, hydrates, solvates or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are each independently selected from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R9R10, P(S)R9R10 and 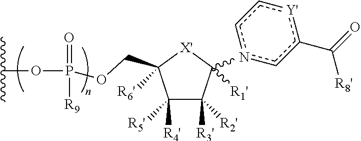
wherein n is an integer equal to 1 or 3; in which
R₉ and R₁₀ are each independently selected from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)C(O)O(C₁-C₁₅)alkyl, and —(CH₂)ₙC(O)O(C₁-C₁₅)allyl aryl; wherein n is an integer selected from 1 to 8; and P(O)(OH)OP(O)(OH)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀, together with the phosphorus atoms to which they are attached, form a 6-membered ring in which —R₉-R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
R₉ and R₁₀, together with the phosphorus atoms to which they are attached, form a 6-membered ring in which —R₉-R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are each independently selected from among H, (C₁-C₈) alkyl and (C₁-C₈) alkyl aryl;
Y is selected from among CH, CH₂, C(CH₃)₂ and CCH₃; [symbol]
represents a single or a double bond along Y; and [symbol]
represents the alpha or beta anomer depending on the position of R₁
or one of the stereoisomers, one of the salts, one of the hydrates, one of the solvates or one of the crystals thereof
or
the compound of formula (Ia): 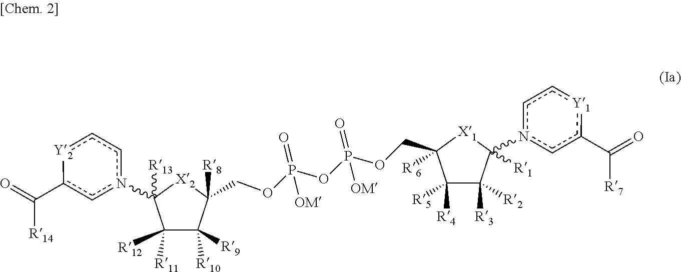
or one of the stereoisomers, one of the salts, one of the hydrates, one of the solvates or one of the crystals thereof, in which
X′₁ and X′₂ are independently selected from among O, CH₂, S, Se, CHF, CF₂, and C═CH₂;
R′₁ and R′13 are independently selected from among H, azido, cyano, a C1-C8 alkyl, a C1-C8 thio-alkyl, a C1-C8 heteroalkyl, and OR, wherein R is selected from H and a C1-C8 alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from among H and a (C₁-C₈) alkyl aryl;
Y′₁ and Y′₂ are independently selected from among CH, CH₂, C(CH₃)₂ or CCH³;
M′ is selected from among H or a suitable counter ion; [symbol]
represents a single or double bond depending on Y′₁ and Y′₂; and [symbol] represents an alpha or beta anomer depending on the position of R′1 and R′13; and combinations thereof.

3. Nicotinamide mononucleotide (NMN), a pharmaceutically acceptable derivative thereof, or a pharmaceutically acceptable salt thereof, for use thereof according to claim 1 in combination with at least one other therapeutic agent.

4. Nicotinamide mononucleotide (NMN), a pharmaceutically acceptable derivative thereof, or a pharmaceutically acceptable salt thereof, for use thereof according to claim 3, wherein the at least one other therapeutic agent is a vaccine that can be selected from among attenuated live vaccines, inactivated vaccines, multivalent vaccines, or combination vaccines.

5. Nicotinamide mononucleotide (NMN), a pharmaceutically acceptable derivative thereof, or a pharmaceutically acceptable salt thereof, for use thereof according to claim 4, wherein the vaccine is selected from among a vaccine against a virus, a bacterium, a parasite, a yeast and/or fungus, or combinations thereof.

6. Nicotinamide mononucleotide (NMN), a pharmaceutically acceptable derivative thereof, or a pharmaceutically acceptable salt thereof, for use thereof according to claim 5, wherein said vaccine is selected from among a vaccine against a virus selected from among Influenzavirus, Coronavirus, Respirovirus, Pneutnovirus, Metapneunovirus, Adenovirus, Enterovirus, Rhinovirus, Hepatovirus, Erbovirus, Aphtovirus, Norovirus, Alphavirus, Rubivirus, Flavivirus, Hepacivirus, Pestivirus, Ebola, Morbillivirus, Rubulavirus, Henipavirus, Arenavirus, Orthobunyavirus, Phlebovirus, Rotavirus, Simiplexvirus, Varicellovirus, Papillomavirus, Cytomegalovirus or combinations thereof.

7. Nicotinamide mononucleotide (NMN), a pharmaceutically acceptable derivative thereof, or a pharmaceutically acceptable salt thereof, for use thereof according to claim 1, wherein the decrease in immunosenescence can be determined by the reduction of a marker selected from among thymic involution, cytokine levels of immunosenescence, the number of resident senescent T cells in the spleen, the level of circulating IgG immunoglobulin produced by memory B cells, the level of circulating IgA immunoglobulin produced by memory B cells, and combinations thereof.

8. Nicotinamide mononucleotide (NMN), a pharmaceutically acceptable derivative thereof, or a pharmaceutically acceptable salt thereof, for use thereof according to claim 1, wherein the decrease in immunosenescence can be determined by the increase of a marker selected from among the production of new naive T cells, the capacity to respond to new antigens, the accumulation of memory T cells, the number of circulating B cells, the level of circulating IgD immunoglobulin produced by naive cells, the level of circulating IgM produced by naive cells, vaccinal immunogenicity and combinations thereof.

9. Nicotinamide mononucleotide (NMN), a pharmaceutically acceptable derivative thereof, or a pharmaceutically acceptable salt thereof, for use thereof according to claim 1 in a form adapted for administering thereof via oral, ocular, sublingual, parenteral, transcutaneous, vaginal, peridural, intravesical, rectal or inhalation route, preferably via oral route.

10. A composition comprising nicotinamide mononucleotide, a pharmaceutically acceptable derivative thereof, or a pharmaceutically acceptable salt thereof, and at least one pharmaceutically acceptable excipient for use thereof according to claim 1.
